(19) United States
(12) Patent Application Publication
(10) Pub. No.: US 2014/0186365 A1
(43) Pub. Date: Jul. 3, 2014

(54) ANTIBODIES THAT SPECIFICALLY BIND TO SERUM ALBUMIN WITHOUT INTERFERING WITH ALBUMIN'S CAPABILITY TO INTERACT WITH THE FCRN

(71) Applicants: Institute for Cancer Research d/b/a The Research Institute of Fox Chase Cancer Center, (US); ONCOBIOLOGICS, INC., Cranbury, NJ (US)

(72) Inventors: Matthew K. Robinson, Blue Bell, PA (US); John K. Cini, Blairstown, NJ (US); Jimson W. D'Souza, Philadelphia, PA (US)

(73) Assignees: Institute for Cancer Research d/b/a The Research Institute of Fox Chase Cancer Center, Philadelphia, PA (US); Oncobiologics, Inc., Cranbury, NJ (US)

(21) Appl. No.: 13/826,656

(22) Filed: Mar. 14, 2013

Related U.S. Application Data
(60) Provisional application No. 61/748,537, filed on Jan. 3, 2013.

(51) Int. Cl. C07K 16/18

(52) U.S. Cl. CPC C07K 16/18
USPC 424/158.1; 530/389.3; 536/23.53; 435/320.1; 435/337; 435/252.3; 435/254.2

(57) ABSTRACT

Antibodies that specifically bind to an epitope on the serum albumin, including human and/or mouse serum albumin are provided. Nucleic acids encoding such antibodies and cells capable of expressing such antibodies are also provided.

### CROSS REFERENCE TO RELATED APPLICATIONS

This application claims priority to U.S. Provisional Application No. 61/748,537 filed on Jan. 3, 2013, the contents of which are incorporated by reference herein, in their entirety and for all purposes.

### REFERENCE TO A SEQUENCE LISTING

This application includes a Sequence Listing submitted electronically as a text file named SEQ_LST_PAT_IN_ST25.txt, created on Mar. 8, 2013 with a size of 86,000 bytes. The Sequence Listing is incorporated by reference herein.

### FIELD OF THE INVENTION

The invention relates generally to the field of immunology. More particularly, the invention relates to antibodies that specifically bind to serum albumin, including human and mouse serum albumin, and methods for using such antibodies as circulating vehicles to capture serum albumin, thereby enhancing the pharmacokinetics of the antibody.

### BACKGROUND OF THE INVENTION

Various publications, including patents, published applications, technical articles, scholarly articles, and gene or protein accession numbers are cited throughout the specification. Each of these materials is incorporated by reference herein, in its entirety and for all purposes.

Chemotherapy regimens represent the widest variety, and the most successful, class of anti-cancer agents for the treatment of a large number of cancers, including, but not limited to, cancers of the breast, colorectal cancer, gastric cancer, head & neck cancer, and lung cancer. The use of biologic molecules, including engineered antibodies and cytokines as chemotherapeutic agents, is often impeded by a relatively short half-life in blood circulation. Accordingly, various strategies have been investigated in an attempt to enhance the circulation half-life of the biologic agents.

Serum albumin exhibits a long circulation half-life. As such, serum albumin is viewed as a desirable partner for biologic chemotherapeutic agents such that these biologic agents may benefit from serum albumin's long circulation half-life. In other words, it is desired to partner a biologic agent with serum albumin such that the biologic agent may share the long circulation half-life of the serum albumin, thereby increasing the circulation half-life of the biologic agent.

It is believed that the interaction of serum albumin with the neonatal Fc receptor (FcRn) on circulating endothelial cells and/or circulating monocytes contributes to its enhanced circulation half-life. It is also believed that this albumin-FcRn connection protects the albumin molecule from intracellular degradation.

Enhancing the pharmacokinetics of chemotherapeutic biologic agents has the potential to enhance their therapeutic efficacy. There is a continuing need for improved pharmacokinetics of biologic agents.

### SUMMARY OF THE INVENTION

The invention provides isolated antibodies that specifically bind to an epitope on serum albumin from any species, with human or mouse serum albumin being preferred. The antibodies may be comprised in a composition comprising a carrier such as a pharmaceutically acceptable carrier. Binding of the antibody to serum albumin increases the pharmacokinetics of the antibody.

The antibodies may comprise a domain antibody, including a heavy chain domain antibody and a light chain domain antibody. A heavy chain domain antibody that specifically binds to an epitope on serum albumin comprises a heavy chain a heavy chain complementarity determining region (CDR) 1 having the amino acid sequence of SEQ ID NO: 146 or SEQ ID NO: 147, a heavy chain CDR2 having the amino acid sequence of SEQ ID NO: 149 or SEQ ID NO: 150, and a heavy chain CDR3 having an amino acid sequence having at least 95% sequence identity with an amino acid sequence selected from the group consisting of SEQ ID NO: 8, SEQ ID NO: 26, SEQ ID NO: 44, SEQ ID NO: 62, SEQ ID NO: 80, SEQ ID NO: 98, SEQ ID NO: 116, and SEQ ID NO: 134. In some aspects, the heavy chain comprises the amino acid sequence of SEQ ID NO: 17, SEQ ID NO: 35, SEQ ID NO: 53, SEQ ID NO: 71, SEQ ID NO: 89, SEQ ID NO: 107, SEQ ID NO: 125, or SEQ ID NO: 143. In some aspects, the heavy chain domain antibody comprises a heavy chain framework (FWR) 1 having the amino acid sequence of SEQ ID NO: 145, a heavy chain FWR2 having the amino acid sequence of SEQ ID NO: 148 or SEQ ID NO:174, a heavy chain FWR3 having the amino acid sequence of SEQ ID NO: 151, and a heavy chain FWR4 having the amino acid sequence of SEQ ID NO: 152.

A light chain domain antibody that specifically binds to an epitope on serum albumin comprises a light chain comprising a light chain CDR1 having the amino acid sequence of SEQ ID NO: 154, SEQ ID NO: 176, SEQ ID NO: 177, SEQ ID NO: 178, SEQ ID NO: 179, or SEQ ID NO: 180, a light chain CDR2 having the amino acid sequence of SEQ ID NO: 156, and a light chain CDR3 having an amino acid sequence of SEQ ID NO: 158, SEQ ID NO: 183, SEQ ID NO: 184, SEQ ID NO: 185, or SEQ ID NO: 186. The light chain may comprise a light chain CDR3 having an amino acid sequence having at least about 95% sequence identity with an amino acid sequence selected from the group consisting of SEQ ID NO: 15, SEQ ID NO: 33, SEQ ID NO: 51, SEQ ID NO: 69, SEQ ID NO: 87, SEQ ID NO: 105, SEQ ID NO: 123, and SEQ ID NO: 141. In some aspects, the light chain comprises the amino acid sequence of SEQ ID NO: 18, SEQ ID NO: 36, SEQ ID NO: 54, SEQ ID NO: 72, SEQ ID NO: 90, SEQ ID NO: 108, SEQ ID NO: 126, or SEQ ID NO: 144. In some aspects, the light chain domain antibody comprises a light chain FWR1 having the amino acid sequence of SEQ ID NO: 153 or SEQ ID NO: 175, a light chain FWR2 having the amino acid sequence of SEQ ID NO: 155, a light chain FWR3 having the amino acid sequence of SEQ ID NO: 157, SEQ ID NO: 181, or SEQ ID NO: 182, and a light chain FWR4 having the amino acid sequence of SEQ ID NO: 159, SEQ ID NO: 187, or SEQ ID NO: 188.

The antibodies may comprise an Fab, or a single chain Fv, or a polyclonal antibody, or a monoclonal antibody. Such an Fab, single chain Fv, polyclonal antibody, or monoclonal antibody comprises a heavy chain comprising a heavy chain FWR1 having the amino acid sequence of SEQ ID NO: 145, a heavy chain CDR1 having the amino acid sequence of SEQ ID NO: 146 or SEQ ID NO: 147, a heavy chain FWR2 having the amino acid sequence of SEQ ID NO: 148 or SEQ ID NO:174, a heavy chain CDR2 having the amino acid sequence of SEQ ID NO: 149 or SEQ ID NO: 150, a heavy chain FWR3 having the amino acid sequence of SEQ ID NO: 151, a heavy chain CDR3 having an amino acid sequence having at least 95% sequence identity with an amino acid sequence selected from the group consisting of SEQ ID NO: 8, SEQ ID NO: 26, SEQ ID NO: 44, SEQ ID NO: 62, SEQ ID NO: 80, SEQ ID NO: 98, SEQ ID NO: 116, and SEQ ID NO: 134, and a heavy chain FWR4 having the amino acid sequence of SEQ ID NO: 152, and comprising a light chain comprising a light chain FWR1 having the amino acid sequence of SEQ ID NO: 153 or SEQ ID NO: 175, a light chain CDR1 having the amino acid sequence of SEQ ID NO: 154, SEQ ID NO: 176, SEQ ID NO: 177, SEQ ID NO: 178, SEQ ID NO: 176, or SEQ ID NO: 180, a light chain FWR2 having the amino acid sequence of SEQ ID NO: 155, a light chain CDR2 having the amino acid sequence of SEQ ID NO: 156, a light chain FWR3 having the amino acid sequence of SEQ ID NO: 157, SEQ ID NO: 181, or SEQ ID NO: 182, a light chain CDR3 having an amino acid sequence of SEQ ID NO: 158, SEQ ID NO: 183, SEQ ID NO: 184, SEQ ID NO: 185, or SEQ ID NO: 186, and a light chain FWR4 having the amino acid sequence of SEQ ID NO: 159, SEQ ID NO: 187, or SEQ ID NO: 188. In some aspects, the heavy chain comprises the amino acid sequence of SEQ ID NO: 17, SEQ ID NO: 35, SEQ ID NO: 53, SEQ ID NO: 71, SEQ ID NO: 89, SEQ ID NO: 107, SEQ ID NO: 125, or SEQ ID NO: 143. In some aspects, the light chain comprises the amino acid sequence of SEQ ID NO: 18, SEQ ID NO: 36, SEQ ID NO: 54, SEQ ID NO: 72, SEQ ID NO: 90, SEQ ID NO: 108, SEQ ID NO: 126, or SEQ ID NO: 144.

The heavy chain domain antibody, light chain domain antibody, Fab, single chain Fv, polyclonal antibody and/or monoclonal antibody preferably has strong affinity for its epitope on the serum albumin molecule. The affinity (K_{d}) may be less than about 1×10⁻⁵ M, less than about 1×10⁻⁶ M, less than about 1×10⁻⁷ M, less than about 1×10⁻⁸ M, less than about 1×10⁻⁹ M, or less than about 1×10⁻¹⁰ M. The epitope preferably is not on a region or domain of the serum albumin molecule that interacts with the neonatal receptor such that binding of the antibody to the epitope preferably does not substantially inhibit the capability of the serum albumin to bind the neonatal receptor when circulating in the blood.

Also provided are polynucleotides encoding such antibodies, as well as vectors comprising such polynucleotides and host cells transformed with such vectors. Such host cells preferably are expression hosts, including mammalian, yeast, insect, and bacterial expression hosts.

### BRIEF DESCRIPTION OF THE DRAWINGS

FIG. 1 shows an alignment of the polypeptide sequences for antibodies to serum albumin, including (top to bottom) antibody 4D6, 3B11, 4D2, 1C5, 3E11, 3G2, 4F2, and 5G8.

FIG. 2 shows sensorgrams generated on Octet® Red 96 (Forte Bio, Delaware, USA) with 4D2 scFv and VH domain antibodies against HSA and MSA. Data was fit to a 1:1 binding model to generate kinetic constants listed in Tables 1 and 2 in the Examples below. FIG. 2A shows representative data on scFv 4D2 characterized against human albumin. The data was fit to 1:1 Langmuir Binding Model to analyze the affinity constants. FIG. 2B shows representative data on heavy chain dAb 4D2 characterized against human albumin. The data was fit to 1:1 Langmuir Binding Model to analyze the affinity constants.

FIG. 3 shows sensorgrams generated on Octet® Red 96 (Forte Bio, Delaware, USA) with 5G8 scFv and VH domain antibodies against HSA and MSA. Data was fit to a 1:1 binding model to generate kinetic constants listed in Tables 1 and 2 in the Examples below. FIG. 3A shows representative data on scFv 5G8 characterized against human albumin. The data was fit to 1:1 Langmuir Binding Model to analyze the affinity constants. FIG. 3B shows representative data on scFv 5G8 characterized against mouse albumin. The data was fit to 1:1 Langmuir Binding Model to analyze the affinity constants. FIG. 3C shows representative data on heavy chain dAb 5G8 characterized against human albumin. The data was fit to 1:1 Langmuir Binding Model to analyze the affinity constants. FIG. 3D shows representative data on heavy chain dAb 5G8 characterized against mouse albumin. The data was fit to 1:1 Langmuir Binding Model to analyze the affinity constants.

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided herein.

The terms subject and patient are used interchangeably and include any animal. Mammals are preferred, including companion and farm mammals, as well as rodents, including mice, rabbits, and rats, and other rodents. Primates are more preferred, and human beings are highly preferred.

A molecule such as an antibody has been “isolated” if it has been altered and/or removed from its natural environment by the hand of a human being.

As used herein, the singular forms “a,” “an,” and “the” include plural referents unless expressly stated otherwise.

An epitope includes an immunological determinant of an antigen that serves as an antibody-binding site. The epitope may be linear or conformational.

Human antibodies that specifically bind to serum albumin with high affinity have been identified in accordance with the invention. Without intending to be limited to any particular theory or mechanism of action, it is believed that these antibodies bind to serum albumin, but do not bind at an epitope on the serum albumin molecule that interferes with the capacity of the serum albumin molecule to bind to the neonatal Fc receptor (FcRn), and it is believed that in binding to serum albumin in this way, the pharmacokinetics of the antibody is enhanced. Accordingly, the invention features antibodies that specifically bind to serum albumin at an epitope that does not significantly interfere, or interfere at all, with the capability of the serum albumin to interact with the FcRn.

The serum albumin may be any serum albumin, but preferably the serum albumin is human albumin and/or mouse albumin. Thus, the antibodies preferably specifically bind to serum albumin, including human serum albumin and/or mouse serum albumin. The antibodies may specifically bind to human serum albumin but not mouse serum albumin, or vice versa, or to bind to both. The antibodies may specifically bind to serum albumin of other species, in addition to human and/or mouse serum albumin, for example, if the antibodies bind to an epitope shared by serum albumin of the different species, including to regions of the serum albumin protein conserved among the species. “HSA” is used to abbreviate human serum albumin. “MSA” is used to abbreviate mouse serum albumin.

The antibodies may comprise any of the five classes of immunoglobulins based on antibody heavy chain structure. For example, the alpha, delta, epsilon, gamma, and mu chains correspond to IgA, IgD, IgE, IgG and IgM isotypes, respectively. The antibodies include all isotypes and synthetic multimers of the four-chain immunoglobulin structure.

The antibodies may be polyclonal, but in some aspects, are not polyclonal. The antibodies preferably are monoclonal. The antibodies may comprise derivatives or fragments or portions of antibodies that retain the antigen-binding specificity, and also preferably retain most or all of the affinity, of the parent antibody molecule. For example, derivatives may comprise at least one variable region (either a heavy chain or light chain variable region). Other examples of suitable antibody derivatives and fragments include, without limitation, antibodies with polyepitopic specificity, bispecific antibodies, diabodies, single-chain molecules, as well as Fab, F(ab′)2, Fd, Fabc, and Fv molecules, single chain (Sc) antibodies, single chain Fv antibodies (scFv), individual antibody light chains, individual antibody heavy chains, fusions between antibody chains and other molecules, heavy chain monomers or dimers, light chain monomers or dimers, dimers consisting of one heavy and one light chain, and other multimers. Single chain Fv antibodies may be multi-valent. All antibody isotypes may be used to produce antibody derivatives, fragments, and portions. Antibody derivatives, fragments, and/or portions may be recombinantly produced and expressed by any cell type, prokaryotic or eukaryotic.

In some preferred aspects, the antibody is a domain antibody (dAb). Domain antibodies include those comprising a single variable antibody domain, including a VH or VL domain, which are able to specifically bind to an antigen. The dAb preferably specifically bind to serum albumin.

In some aspects, a heavy chain domain antibody specifically binds to an epitope on serum albumin. The dAb comprises a heavy chain, which preferably comprises four framework regions (FWR), including FWR1, FWR2, FWR3, and FWR4, and three complementarity determining regions (CDR), including CDR1, CDR2, and CDR3. In some detailed aspects, the heavy chain FWR1 comprises the amino acid sequence of SEQ ID NO:145, the heavy chain FWR2 comprises the amino acid sequence of SEQ ID NO:148 or SEQ ID NO: 174, the heavy chain FWR3 comprises the amino acid sequence of SEQ ID NO:151, and the heavy chain FWR 4 comprises the amino acid sequence of SEQ ID NO:152. In some detailed aspects, the heavy chain CDR1 comprises the amino acid sequence of SEQ ID NO:146 or SEQ ID NO:147, the heavy chain a heavy chain CDR2 comprises the amino acid sequence of SEQ ID NO:149 or SEQ ID NO:150, and the heavy chain CDR3 comprises an amino acid sequence having at least about 85%, at least about 90%, at least about 95%, at least about 96%, at least about 97%, at least about 98%, at least about 99%, or at least about 100% sequence identity with the amino acid sequence of SEQ ID NO:8, SEQ ID NO:26, SEQ ID NO:44, SEQ ID NO:62, SEQ ID NO:80, SEQ ID NO:98, SEQ ID NO:116, or SEQ ID NO:134. Heavy chain dAb variants comprising a CDR3 having an amino acid sequence with less than about 100% identify with the specified sequence identifier retain serum albumin specific binding activity. The retained serum albumin specific binding activity (including affinity) is preferably about the same as the binding activity (including affinity) of a dAb comprising a CDR3 having an amino acid sequence with 100% identity with the specific sequence identifier, although the binding activity (including affinity) may be lesser or greater than the dAb comprising a CDR3 having an amino acid sequence with 100% identity with the specific sequence identifier.

In some preferred aspects, a heavy chain domain antibody may comprise a heavy chain having an amino acid sequence having at least about 85%, at least about 90%, at least about 95%, at least about 96%, at least about 97%, at least about 98%, at least about 99%, or at least about 100% sequence identity with the amino acid sequence of SEQ ID NO:17, SEQ ID NO:35, SEQ ID NO:53, SEQ ID NO:71, SEQ ID NO:89, SEQ ID NO:107, SEQ ID NO: 125, or SEQ ID NO:143. Heavy chain dAb variants comprising a heavy chain having an amino acid sequence with less than about 100% identify with the specified sequence identifier retain serum albumin specific binding activity. The retained serum albumin specific binding activity (including affinity) is preferably about the same as the binding activity (including affinity) of a dAb comprising a heavy chain having an amino acid sequence with 100% identity with the specific sequence identifier, although the binding activity (including affinity) may be lesser or greater than the dAb comprising a heavy chain having an amino acid sequence with 100% identity with the specific sequence identifier.

In some aspects, a light chain domain antibody specifically binds to an epitope on serum albumin. The dAb comprises a heavy chain, which preferably comprises four framework regions (FWR), including FWR1, FWR2, FWR3, and FWR4, and three complementarity determining regions (CDR), including CDR1, CDR2, and CDR3. In some detailed aspects, the light chain FWR1 comprises the amino acid sequence of SEQ ID NO:153 or SEQ ID NO:175, the light chain FWR2 comprises the amino acid sequence of SEQ ID NO:155, the light chain FWR3 comprises the amino acid sequence of SEQ ID NO:157, SEQ ID NO: 181, or SEQ ID NO: 182, and the light chain FWR4 comprises the amino acid sequence of SEQ ID NO:159, SEQ ID NO: 187, or SEQ ID NO: 188. In some detailed aspects, the light chain CDR1 comprises the amino acid sequence of SEQ ID NO:154, SEQ ID NO: 176, SEQ ID NO: 177, SEQ ID NO: 178, SEQ ID NO: 179, or SEQ ID NO: 180, the light chain CDR2 comprises the amino acid sequence of SEQ ID NO:156, and the light chain CDR3 comprises the amino acid sequence of SEQ ID NO:158, SEQ ID NO: 183, SEQ ID NO: 184, SEQ ID NO: 185, or SEQ ID NO: 186. In some aspects, the light chain CDR3 comprises an amino acid sequence having at least about 85%, at least about 90%, at least about 95%, at least about 96%, at least about 97%, at least about 98%, at least about 99%, or at least about 100% sequence identity with the amino acid sequence of SEQ ID NO:15, SEQ ID NO:33, SEQ ID NO:51, SEQ ID NO:69, SEQ ID NO:87, SEQ ID NO:105, SEQ ID NO:123, or SEQ ID NO:141. Light chain dAb variants comprising a CDR3 having an amino acid sequence with less than about 100% identify with the specified sequence identifier retain serum albumin specific binding activity. The retained serum albumin specific binding activity (including affinity) is preferably about the same as the binding activity (including affinity) of a dAb comprising a CDR3 having an amino acid sequence with 100% identity with the specific sequence identifier, although the binding activity (including affinity) may be lesser or greater than the dAb comprising a CDR3 having an amino acid sequence with 100% identity with the specific sequence identifier.

In some preferred aspects, a light chain domain antibody may comprise a light chain having an amino acid sequence having at least about 85%, at least about 90%, at least about 95%, at least about 96%, at least about 97%, at least about 98%, at least about 99%, or at least about 100% sequence identity with the amino acid sequence of SEQ ID NO:18, SEQ ID NO:36, SEQ ID NO:54, SEQ ID NO:72, SEQ ID NO:90, SEQ ID NO:108, SEQ ID NO: 126, or SEQ ID NO:144. Light chain dAb variants comprising a light chain having an amino acid sequence with less than about 100% identify with the specified sequence identifier retain serum albumin specific binding activity. The retained serum albumin specific binding activity (including affinity) is preferably about the same as the binding activity (including affinity) of a dAb comprising a light chain having an amino acid sequence with 100% identity with the specific sequence identifier, although the binding activity (including affinity) may be lesser or greater than the dAb comprising a light chain having an amino acid sequence with 100% identity with the specific sequence identifier.

Domain antibodies may be fused to other polypeptides, including antibody Fc domains such as human IgG1 Fc domains (e.g., SEQ ID NO:163 or SEQ ID NO:164), or IgG4 Fc domains (e.g., SEQ ID NO:165 or SEQ ID NO:166) in order to further enhance their circulating half-life. In some aspects, the combination of the Fc domain and serum albumin-binding further enhances the pharmacokinetics of the antibody.

The antibodies may comprise a single chain Fv molecule (scFv), Fab, or full IgG. Any such antibodies may comprise a heavy chain having an amino acid sequence having at least about 85%, at least about 90%, at least about 95%, at least about 96%, at least about 97%, at least about 98%, at least about 99%, or at least about 100% sequence identity with the amino acid sequence of SEQ ID NO:17, SEQ ID NO:35, SEQ ID NO:53, SEQ ID NO:71, SEQ ID NO:89, SEQ ID NO:107, SEQ ID NO: 125, or SEQ ID NO:143. Antibodies comprising heavy chain variants comprising a heavy chain having an amino acid sequence with less than about 100% identify with the specified sequence identifier retain serum albumin specific binding activity. The retained serum albumin specific binding activity (including affinity) is preferably about the same as the binding activity (including affinity) of an antibody comprising a heavy chain having an amino acid sequence with 100% identity with the specific sequence identifier, although the binding activity (including affinity) may be lesser or greater than the antibody comprising a heavy chain having an amino acid sequence with 100% identity with the specific sequence identifier. The antibody may comprise a light chain having an amino acid sequence having at least about 85%, at least about 90%, at least about 95%, at least about 96%, at least about 97%, at least about 98%, at least about 99%, or at least about 100% sequence identity with the amino acid sequence of SEQ ID NO:18, SEQ ID NO:36, SEQ ID NO:54, SEQ ID NO:72, SEQ ID NO:90, SEQ ID NO:108, SEQ ID NO: 126, or SEQ ID NO:144. Light chain antibody variants comprising a light chain having an amino acid sequence with less than about 100% identify with the specified sequence identifier retain serum albumin specific binding activity. The retained serum albumin specific binding activity (including affinity) is preferably about the same as the binding activity (including affinity) of an antibody comprising a light chain having an amino acid sequence with 100% identity with the specific sequence identifier, although the binding activity (including affinity) may be lesser or greater than the antibody comprising a light chain having an amino acid sequence with 100% identity with the specific sequence identifier.

In some aspects, the antibody comprises particular heavy and light chain pairs. The heavy chains having the amino acid sequences of SEQ ID NO:17, SEQ ID NO:35, SEQ ID NO:53, SEQ ID NO:71, SEQ ID NO:89, SEQ ID NO:107, SEQ ID NO: 125, or SEQ ID NO:143 may be paired with any light chains having the amino acid sequences of SEQ ID NO:18, SEQ ID NO:36, SEQ ID NO:54, SEQ ID NO:72, SEQ ID NO:90, SEQ ID NO:108, SEQ ID NO: 126, or SEQ ID NO:144. Preferred pairs include SEQ ID NO:17 and SEQ ID NO:18, SEQ ID NO:35 and SEQ ID NO:36, SEQ ID NO:53 and SEQ ID NO:54, SEQ ID NO:71 and SEQ ID NO:72, SEQ ID NO:89 and SEQ ID NO:90, SEQ ID NO:107 and SEQ ID NO:108, SEQ ID NO:125 and SEQ ID NO:126, SEQ ID NO:143 and SEQ ID NO:144. In some preferred aspects, the antibodies comprise a scFv comprising a heavy and light chain variable region comprising the amino acid sequence of SEQ ID NO:2, SEQ ID NO:20., SEQ ID NO:38, SEQ ID NO:56, SEQ ID NO:74, SEQ ID NO:92, SEQ ID NO:110, or SEQ ID NO:28.

Single chain Fv heavy and light chain pairs may be fused to each other via a linker, including Gly-Ser linkers. A preferred linker comprises a Gly₄-Ser₁ repeat, including one, two, three, four, five or more repeats of the glycine-serine sequence, e.g., SEQ ID NO:168-171. In some aspects, the linker comprises only a single Gly₄-Ser₁ linking peptide, e.g., SEQ ID NO:167. The linker may comprise a lysine residue, e.g., SEQ ID NO:172 or SEQ ID NO:173.

Natural sequence variations may exist among heavy and light chains and the genes encoding them, and therefore the person having ordinary skill in the art would expect to find some level of variation within the amino acid sequences, or the genes encoding them, of the antibodies described and exemplified herein. These variants preferably maintain the unique binding properties (e.g., specificity and affinity) of the parent antibody. Such an expectation is due in part to the degeneracy of the genetic code, as well as to the known evolutionary success of conservative amino acid sequence variations, which do not appreciably alter the nature of the encoded protein. Accordingly, such variants and homologs are considered substantially the same as one another and are included within the scope of the present invention. The antibodies thus include variants having single or multiple amino acid substitutions, deletions, additions, or replacements that retain the biological properties (e.g., binding specificity and binding affinity) of the parent antibodies. The variants are preferably conservative, but may be non-conservative.

The antibodies may comprise post-translational modifications or moieties, which may impact antibody activity or stability. These modifications or moieties include, but are not limited to, methylated, acetylated, glycosylated, sulfated, phosphorylated, carboxylated, and amidated moieties and other moieties that are well known in the art. Moieties include any chemical group or combinations of groups commonly found on immunoglobulin molecules in nature, or otherwise added to antibodies by recombinant expression systems, including prokaryotic and eukaryotic expression systems.

The antibodies may be derived from any species. For example, the antibodies may be mouse, rat, goat, horse, swine, bovine, camel, chicken, rabbit, donkey, llama, dromedary, shark, or human antibodies, as well as antibodies from any other animal species. For use in the treatment of humans, non-human derived antibodies may be structurally altered to be less antigenic upon administration to a human patient, including by chimerization or humanization.

Thus, in some aspects, the antibodies are chimeric antibodies. Chimeric antibodies include portions from different species. For example, a chimeric antibody may comprise a mouse antigen binding domain coupled to a human Fc domain or other such structural domain. Preferred chimeric antibodies include heavy and light chain variable regions not of human origin and constant regions of human origin. Chimeric antibodies and methods to produce them are well known and established in the art.

In some aspects, the antibodies are humanized antibodies. Humanized antibodies are those wherein the amino acids directly involved in antigen binding, e.g., the complementarity determining regions (CDR), and in some cases the framework regions (FWR), or portions thereof, of the heavy and/or light chains are not of human origin, while the rest of the amino acids in the antibody are human or otherwise of human origin, e.g., a human antibody scaffold. The antibodies may be humanized chimeric antibodies. Direct involvement in antigen binding includes direct participation in the interactions of antibody amino acids with the epitope, as well as indirect participation such as the effects on structural aspects of the antibody combining site that allow other amino acids to be oriented in a position where they are able to directly participate in the interactions with the epitope.

In highly preferred aspects, the antibodies are fully human. Fully human antibodies are those where the whole molecule is human or otherwise of human origin, or includes an amino acid sequence identical to a human form of the antibody. Fully human antibodies include those obtained from a human V gene library, for example, where human genes encoding variable regions of antibodies are recombinantly expressed. Fully human antibodies may be expressed in other organisms (e.g., mice and xenomouse technology) or cells from other organisms transformed with genes encoding human antibodies.

The antibodies may be labeled or conjugated to any chemical or biomolecule moieties. Labeled antibodies may find use in therapeutic, diagnostic, or basic research applications. Such labels/conjugates can be detectable, such as fluorochromes, radiolabels, enzymes, fluorescent proteins, and biotin. The labels/conjugates may be chemotherapeutic agents, toxins, isotopes, and other agents used for treating conditions such as the killing of cancer cells. Chemotherapeutic agents may be any suitable for the purpose to which the antibody is being used. In the case of treating tumors, the agent may be among the class of alkylating agents, antimetabolites, anthracyclines, antibiotics, platinums, plant alkaloids, vinca alkaloids, topoisomerase inhibitors, taxanes, hormones, corticosteroids, epipodophyllotoxins, maytansanoids, auristatin derivatives, and other agents known or used to treat any aspect of tumor growth, sustenance, or proliferation, including the killing of the tumor cells or inhibition of angiogenesis or neovascularization of a tumor.

The antibodies may be derivatized by known protecting/blocking groups to prevent proteolytic cleavage or enhance activity or stability.

The antibodies preferably have a binding affinity for an epitope on serum albumin that includes a dissociation constant (K_{d}) of less than about 1×10⁻² M. In some embodiments, the K_{d} is less than about 1×10⁻³ M. In other embodiments, the K_{d} is less than about 1×10⁻⁴ M. In some embodiments, the K_{d} is less than about 1×10⁻⁵ M. In still other embodiments, the K_{d} is less than about 1×10⁻⁶ M. In other embodiments, the K_{d} is less than about 1×10⁻² M. In other embodiments, the K_{d} is less than about 1×10⁻⁸ M. In other embodiments, the K_{d} is less than about 1×10⁻⁹ M. In other embodiments, the K_{d} is less than about 1×10⁻¹⁰ M. In still other embodiments, the K_{d} is less than about 1×10⁻¹¹ M. In some embodiments, the K_{d} is less than about 1×10⁻¹² M. In other embodiments, the K_{d} is less than about 1×10⁻¹³ M. In other embodiments, the K_{d} is less than about 1×10⁻¹⁴ M. In still other embodiments, the K_{d} is less than about 1×10⁻¹⁵ M. Affinity values refer to those obtained by standard methodologies, including surface plasmon resonance such as Biacore™ analyses or analysis using an Octet® Red 96 (Forte Bio) Dip-and-Read system.

Polynucleotide sequences that encode antibodies and their subdomains (e.g., FWRs and CDRs) are featured in the invention. Polynucleotides include, but are not limited to, RNA, DNA, hybrids of RNA and DNA, and single, double, or triple stranded strands of RNA, DNA, or hybrids thereof.

In some aspects, the polynucleotides encode the heavy chain of an antibody that specifically binds to an epitope on serum albumin. The polynucleotide may encode a heavy chain comprising the amino acid sequence of any of SEQ ID NO:17, SEQ ID NO:35, SEQ ID NO:53, SEQ ID NO:71, SEQ ID NO:89, SEQ ID NO:107, SEQ ID NO: 125, or SEQ ID NO:143. The polynucleotide may encode a light chain comprising the amino acid sequence of any of SEQ ID NO:18, SEQ ID NO:36, SEQ ID NO:54, SEQ ID NO:72, SEQ ID NO:90, SEQ ID NO:108, SEQ ID NO: 126, or SEQ ID NO:144. The polynucleotide may comprise the nucleic acid sequence of any of SEQ ID NO:1, SEQ ID NO:19, SEQ ID NO:37, SEQ ID NO:55, SEQ ID NO:73, SEQ ID NO:91, SEQ ID NO: 109, or SEQ ID NO:127. The polynucleotide may comprise a nucleic acid sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity with any of SEQ ID NO:1, SEQ ID NO:19, SEQ ID NO:37, SEQ ID NO:55, SEQ ID NO:73, SEQ ID NO:91, SEQ ID NO: 109, or SEQ ID NO:127, and in some aspects such variants preferably encode the same amino acids encoded by the polynucleotide sequence of SEQ ID NO:1, SEQ ID NO:19, SEQ ID NO:37, SEQ ID NO:55, SEQ ID NO:73, SEQ ID NO:91, SEQ ID NO: 109, or SEQ ID NO:127. Preferably, the antibodies encoded by the polynucleotide variants will specifically bind to an epitope on serum albumin with an affinity about equal to the affinity of the antibody encoded by the parent (non-variant) polynucleotide sequence. Complements of the polynucleotide sequences and the variant polynucleotide sequences are also within the scope of the invention.

Also encompassed within the invention are vectors comprising the polynucleotides of the invention. The vectors may be expression vectors. Recombinant expression vectors containing a sequence encoding a polypeptide of interest are thus provided. The expression vector may contain one or more additional sequences, such as but not limited to regulatory sequences, a selection marker, a purification tag, or a polyadenylation signal. Such regulatory elements may include a transcriptional promoter, enhancers, mRNA ribosomal binding sites, or sequences that control the termination of transcription and translation.

Expression vectors, especially mammalian expression vectors, may include one or more nontranscribed elements, such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a specific host may also be incorporated.

The vectors may be used to transform any of a wide array of host cells well known to those of skill in the art, and preferably host cells capable of expressing antibodies. Vectors include without limitation, plasmids, phagemids, cosmids, baculoviruses, bacmids, bacterial artificial chromosomes (BACs), yeast artificial chromosomes (YACs), and baculovirus, as well as other bacterial, eukaryotic, yeast, and viral vectors. Suitable host cells include without limitation CHO cells, HEK293 cells, or any eukaryotic stable cell line known or produced, and also include bacteria, yeast, and insect cells.

The antibodies may also be produced by hybridoma cells; methods to produce hybridomas being well known and established in the art.

The invention also features compositions. The compositions may comprise any of the antibodies described and/or exemplified herein and an acceptable carrier such as a pharmaceutically acceptable carrier. Suitable carriers include any media that does not interfere with the biological activity of the antibody and preferably is not toxic to a host to which it is administered. The carrier may be an aqueous solution, such as water, saline, or alcohol, or a physiologically compatible buffer, such as Hanks's solution, Ringer's solution, or physiological saline buffer. The carrier may contain formulatory agents, such as suspending, stabilizing and/or dispersing agents.

The compositions may also be formulated in sustained release vehicles or depot preparations. For example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well-known examples of delivery vehicles suitable for use as carriers for hydrophobic drugs.

Preferably, any antibody described or exemplified herein preferably specifically binds to serum albumin at an epitope on the serum albumin molecule that does not participate in the interaction of the serum albumin molecule with the FcRn. Binding of the antibody to the serum albumin molecule thus preferably does not substantially interfere with, inhibit, prevent, or otherwise reduce binding of the serum albumin molecule with the FcRn. Preferably, the antibody does not compete with the FcRn for binding to the serum albumin molecule. Preferably, the antibody does not sterically inhibit binding of serum albumin to the FcRn. Preferably, the antibody does not change the conformation of the serum albumin molecule such that the albumin cannot interact with the FcRn.

The invention also features methods for enhancing the pharmacokinetics, including but not limited to the circulating half-life, of an antibody that specifically binds to serum albumin. The methods may be carried out in vitro, in vivo, or in situ.

In some aspects, the methods comprise contacting an antibody described or exemplified herein, or a composition comprising the antibody, with serum albumin in the blood of a subject such that the antibody specifically binds to the serum albumin, thereby enhancing the circulating half-life of the antibody. Preferably, the antibody does not substantially interfere with, inhibit, prevent, or otherwise reduce binding of the serum albumin molecule with the FcRn.

In some aspects, the methods comprise administering an antibody described or exemplified herein, or a composition comprising the antibody, to the blood of a subject, such that the antibody specifically binds to serum albumin in the blood, thereby enhancing the circulating half-life of the antibody. Administering the antibody may be according to any means suitable in the art, including by injection or intravenous infusion. Preferably, the antibody does not substantially interfere with, inhibit, prevent, or otherwise reduce binding of the serum albumin molecule with the FcRn.

The invention also features kits comprising the antibodies described and exemplified herein. The kits may be used to supply antibodies and other agents for use in diagnostic, basic research, or therapeutic methods, among others.

In some aspects, a kit comprises an antibody that specifically binds to an epitope on serum albumin and instructions for using the kit in a method for enhancing the pharmacokinetics of the antibody. The kits may comprise a pharmaceutically acceptable carrier. In the kits, the antibody may be any antibody described or exemplified herein.

The following examples are provided to describe the invention in greater detail. They are intended to illustrate, not to limit, the invention.

### EXAMPLE 1

### Panning and Selection of HSA-Binding scFv Antibodies

An scFv phagemid library (Yuan, et al. (2008) Caner Immunol. Immunother. 57:367-378) was expressed and used to select antibodies specific for human serum albumin. Soluble HSA at 100 μg/ml concentration in BupH Carbonate-Bicarbonate buffer was coated onto immunotubes overnight at 4° C. The immunotube was rinsed with phosphate buffered saline (PBS) and blocked with 2% gelatin in PBS for 2 hours at room temperature.

Purified phage (at a diversity of 10¹⁰ and titer of 10¹³ cfu/ml) were mixed with 1 ml of 1% gelatin-PBS and added to the blocked immunotube. The mixture was incubated in the immunotube for 1.5 hours (rotation for 30 min and stationary for 90 min) at room temperature. Unbound phage particles were discarded followed by washing the immunotube 5 times with PBST (PBS containing 0.1% v/v Tween 20), then 5 times with PBS. Bound phage particles were eluted with 1 ml of freshly prepared 100 mM TEA by rotating the immunotube for 10 min at room temperature. The eluted phage were transferred to a fresh tube and neutralized with 0.5 ml of 1 M Tris-HCl, pH 7.4.

Half of the neutralized elute (0.75 ml) was used to infect 20 ml of exponentially growing E. coli TG1 cells. After infection for 30 min in a 30° C. waterbath with occasional shaking, the infected cells were grown on 2× YTCG plates (2× YT with 25 ug/ml Chloramphenicol and 2% Glucose) overnight at 30° C. Colonies were scraped off the plates and harvested in 10 ml 2× YTCG and to that 4.5 ml of 50% Glycerol was added for storage in −80° C. In order to rescue the scFv-phage particles, 260 μl of glycerol stock bacteria were inoculated in 100 ml 2× YTCG in a 250 ml baffled flask and incubated with agitation in a shaker at 30° C. until the culture reached a log phase. 10 ml of exponentially growing culture was superinfected with Hyperphage M13KO7ΔpIII (Progen, Cat. #PRHYPE-XS) at a multiplicity of infection 1:15 and incubated for 1 hour at 30° C. (30 min stationary and 30 min on shaker).

To determine the efficiency of helper phage infection, 1 μl of the infected culture was diluted in 1 ml 2× YT media and plated on 2×YTCG and 2× YTKG (2× YT with 30 ug/ml Kanamycin and 2% Glucose). The remaining culture was centrifuged at 3000 rpm for 20 min and gently resuspended in 50 ml 2× YTCGI (2× YT with 25 ug/ml Chloramphenicol, 2% Glucose and 0.5 mM IPTG). The phage production was induced overnight at 30° C. in a shaker. In the following step, the culture was centrifuged at 4000 rpm for 45 min at 4° C. and ⅕th volume of PEG-NaCl (20% PEG 6000, 2.5 M NaCl) was added to the phage-containing supernatant. The mixture was cooled on ice for 1 hour and centrifuged at 4000 rpm for 30 min at 4° C.

A white pellet of phage particles was isolated and reconstituted in 1 ml cold PBS. After reconstituting, the remaining bacterial debris was separated by an additional spin at 13000 rpm for 5 min at 4° C. A 2 μl sample of concentrated scFv-phage particles was used for titration. The isolated scFv-phage were directly used for subsequent rounds of selection or stored in 50% Glycerol at −80° C. A total three rounds of selection were performed during the entire screen for selecting binders to HSA. The concentration of HSA that was used to coat the immunotubes remained 100 μg/ml during the subsequent rounds of panning but the number of washes was increased by 5 for each round.

### EXAMPLE 2

### Specificity of Antibodies for HSA by a Phage ELISA

To determine the specificity of antibodies for HSA, a phage ELISA was performed against soluble HSA and control proteins. Individual E. coli colonies from round 3 of selection were picked into 96-well plates containing 200 μl of 2× YTCG medium per well. The plates were incubated overnight at 30° C. in a shaker and they served as Master Plates for Phage ELISA. A 5 μl of inoculum from Master Plates was transferred to Working Plates containing 180 μl of fresh 2× YTCG medium per well and incubated in a shaker at 30° C. for 1.5 hours. A 50 μl volume of Hyperphage (at 1012 cfu/ml) was diluted in 15 ml of fresh 2× YT media and 15 μl per well was used to infect exponentially growing E. coli TG1 cells. The plates were incubated for additional 1.5 hours (30 min stationary and 60 min shaking) at 30° C. followed by centrifugation at 1200 rpm for 20 min and the supernatant was carefully discarded. Fresh 2× YTCGI was added (200 μl per well) and the plates were incubated overnight in a shaker at 30° C. The next day, plates were centrifuged at 2000 rpm for 20 min and 50 μl of phage-containing supernatant from each well was preblocked with 50 μl of 1% Gelatin and used for Phage ELISA.

Human Serum Albumin and control proteins at 100 μg/ml were coated overnight at 4° C. onto Maxisorp® 96-well microtiter plates in BupH Carbonate-Bicarbonate buffer. After coating, the solution was discarded from the wells and the plates were blocked overnight at 4° C. with 1% Gelatin-PBS. The next day, plates were rinsed with PBS and 100 μl of the preblocked Phage were added to each well. The plates were incubated at room temperature for 2 hours and then washed 3 times with PBST. To each well, 100 μl of a 1:2000 dilution of anti-M13 HRP-conjugated antibody (Creative Biomart, Cat. #CAB-655M) in 1% Gelatin-PBS was added and the plates were incubated for 2 hours. Each plate was washed 3 times with PBST and then 3 times with PBS and 100 μl of TMB substrate (KPL, Cat. #53-00-02) was added to each well and samples were incubated for approximately 10 min or until they developed color. The reaction was stopped with 100 μl of 1 M HCl. The signal generated was measured by reading the absorbance at 450 nm using a microtiter plate reader. Clones showing specific binding to HSA were picked based on apparent absorbance values in reference to the control wells and were subjected to repeated ELISA in triplicates for confirmation.

### EXAMPLE 3

### Identification of Antibody Clones

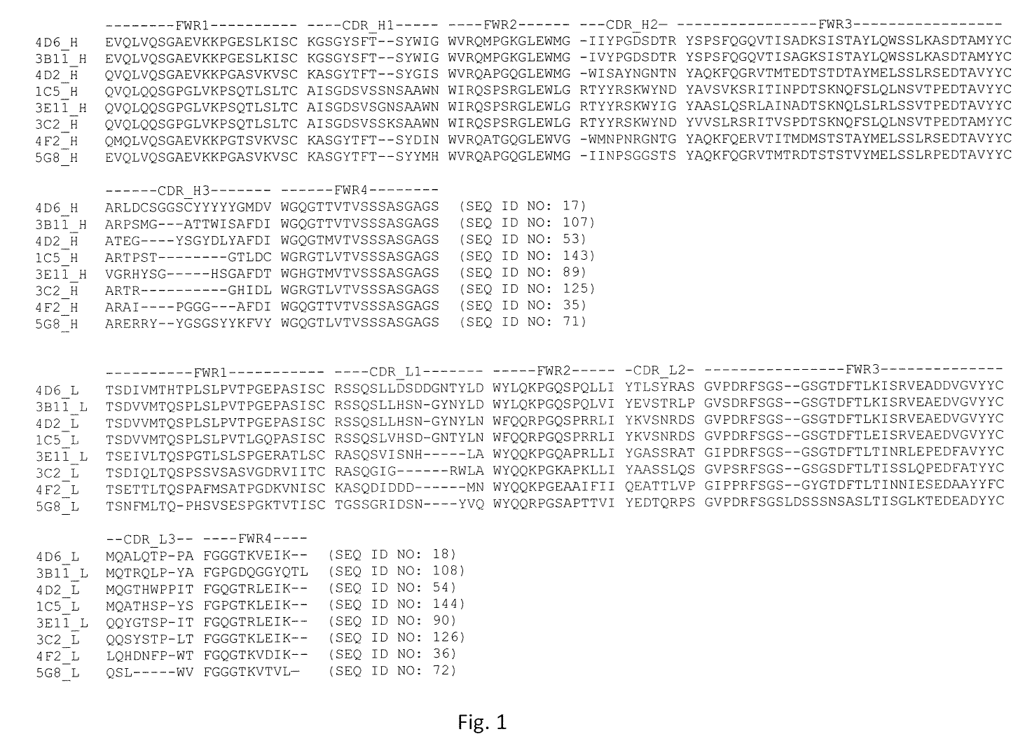

The clones that were identified to be positive for specific binding to HSA by Phage ELISA were sequenced with LW 111 (5′-CAGGAAACAGCTATGAC-3′) (SEQ ID NO:160) that reads the VH region of the scFv. The aligned antibody sequences are shown in FIG. 1. Sequencing analysis revealed eight unique clones of scFv antibodies against HSA. Further comparison of antibody sequences with the germline sequences in IMGT database showed that the VH regions corresponding to scFvs 1C5, 3C2 and 3E11 were derived from gene IGHV6-1, the VH regions that corresponded to scFvs 3B11 and 4D6 were derived from gene IGHV5-51 while the VH regions corresponding to scFv 5G8 was derived from IGHV1-46, 4D2 from gene IGHV1-18 and 4F2 from gene IGHV1-8.

### EXAMPLE 4

### Expression, Purification and Characterization of scFv Antibodies

The scFv antibody coding fragments were restricted from the phagemid vector pAK100-Ink with NcoI and NotI for directional subcloning into a bacterial expression vector with an N-terminal cleavable PeIB leader sequence for periplasmic expression and a C-terminal 6× His-tag for IMAC purification. The clones were verified for correct orientation of ORF's by sequencing with LW 111 and LW 196 (5′-GTTGGGAAGGGCGATCGG-3′) (SEQ ID NO:161). Soluble scFv's were expressed in E. coli TG1 cells by overnight induction with 0.5 mM IPTG at 18° C. Crude periplasmic extract was isolated by reconstituting the bacterial pellets in 20% (w/v) Sucrose, 50 mM Tris-HCl, pH 8.0, 1 mM EDTA, pH 8.0 and cooled on ice for 20 min. Cellular debris was removed by centrifugation at 12000 rpm for 50 min and the periplasmic extract was subjected to overnight dialysis against PBS at 4° C. The next day, scFv dialysate was run on Ni-NTA agarose column for affinity purification. The size and integrity of the resulting scFv were assayed by 12% SDS PAGE under reducing conditions.

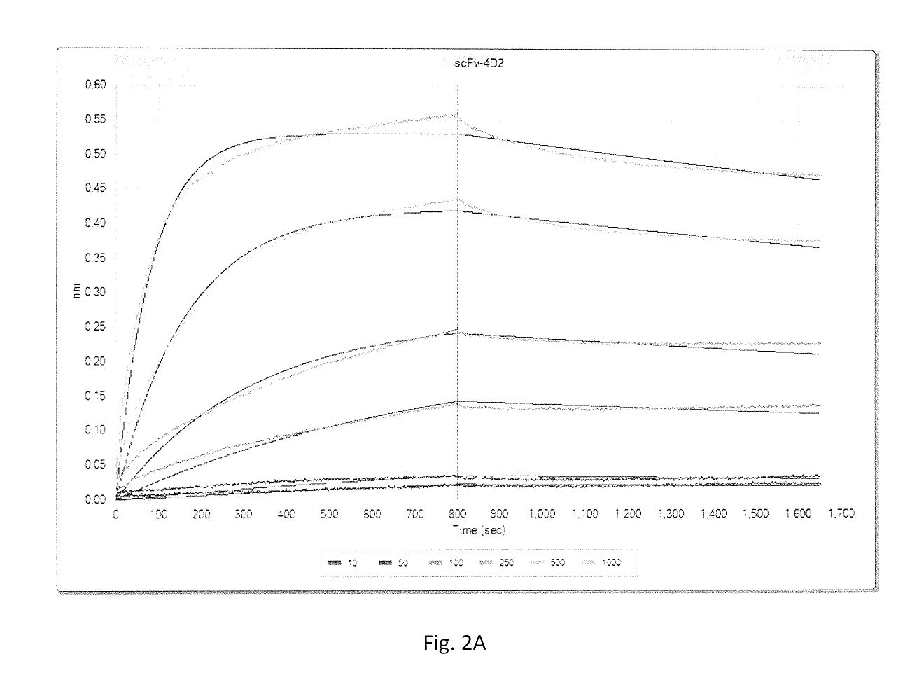

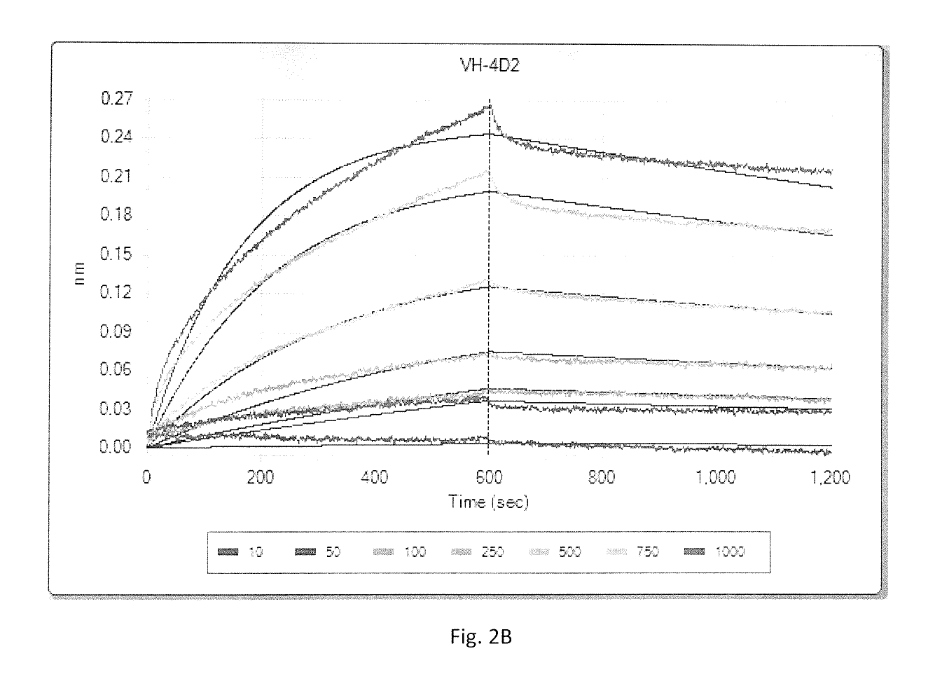

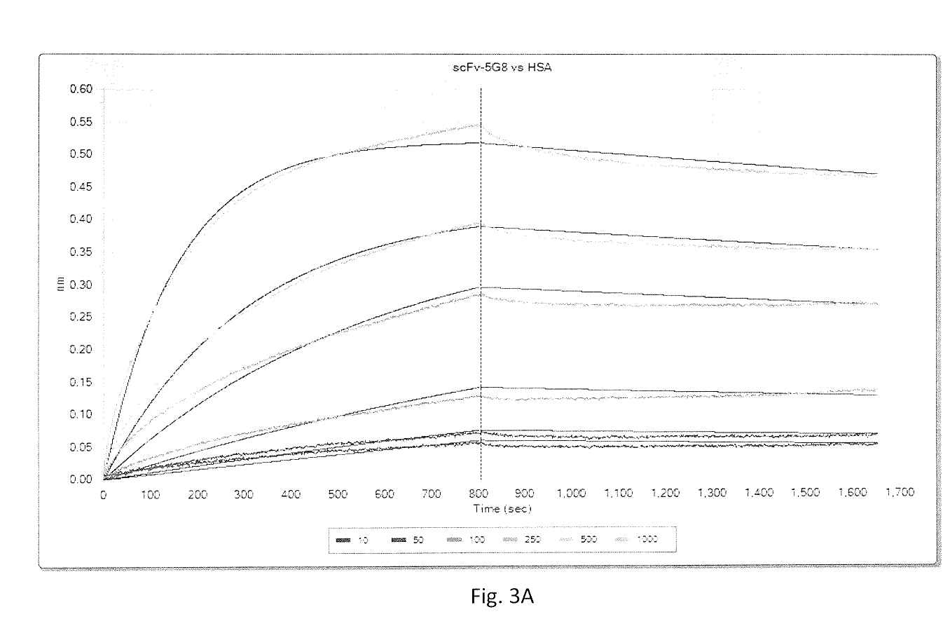

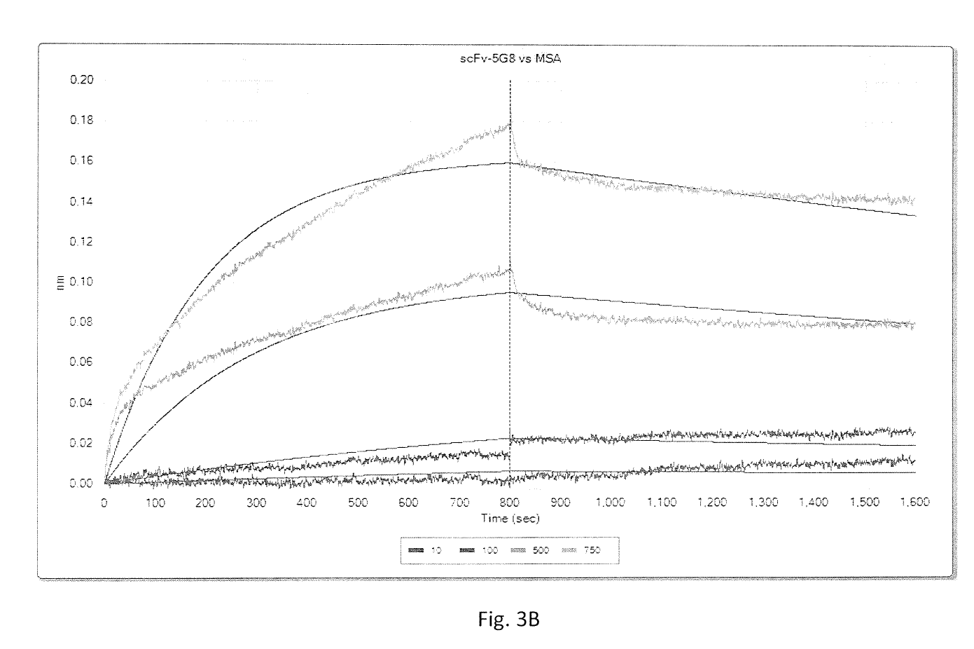

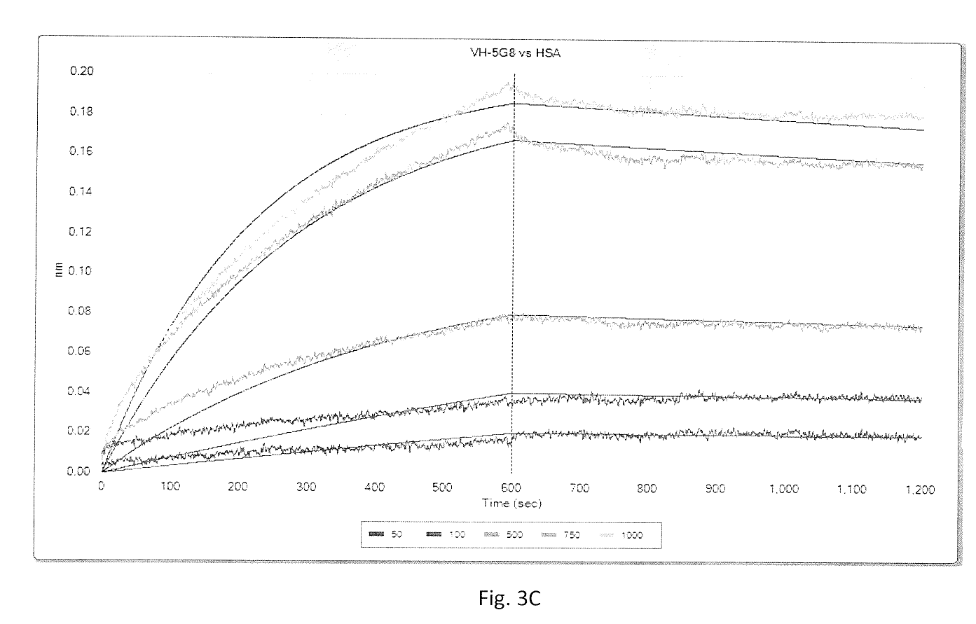

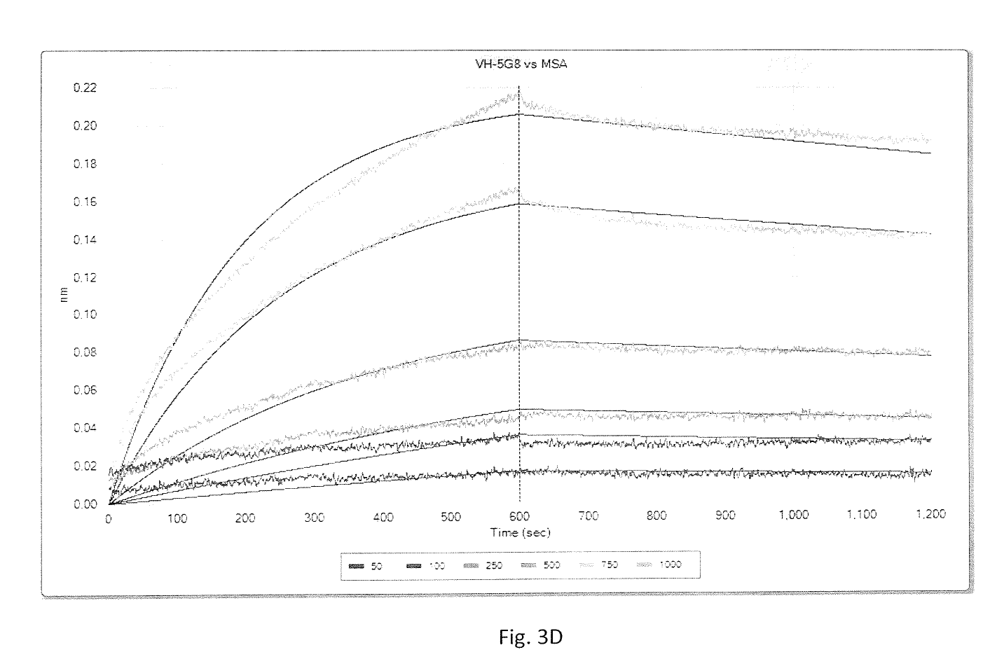

The purified scFv antibodies were further analyzed on Octet Red 96 (Forte Bio) Dip-and-Read system for determining their binding affinity. Human Serum Albumin was biotinylated at a ratio of 1:2 (HSA:Biotin) and was loaded onto the pre-wet Streptavidin-coated sensor tips at 3 μg/ml for 60 sec to achieve a response of approximately 0.3-0.4 nm. After obtaining a steady baseline, the sensors were dipped into varying concentrations of scFv diluted in PBS. The association and dissociation times were set to 600 sec. The data was processed with Data Analysis software v. 7 followed by global fitting of all the response curves. The curves were fitted to a simple 1:1 Langmuir binding model (A+B⇄AB) to calculate the on-rate (Ka), off-rate (Kd) and the equilibrium dissociation constant (Kd/Ka=KD). In a subsequent set of experiments, the scFv antibodies were tested for cross-reactivity with Mouse Serum Albumin. The experimental conditions were kept similar and consistent with those for HSA. The binding affinity of scFv to HSA and cross-reactivity to MSA was determined with an Octet Red system (FIG. 2A, FIG. 3A, and FIG. 3B). The affinity constants for each of the scFv antibodies are represented in Table 1.

TABLE 1
scFv antibody binding to human (HSA) and mouse serum albumin (MSA).

| | Yields | HSA | | | MSA | | |
|---|---|---|---|---|---|---|---|
| | μg/L | Kₒₙ (1/Ms) | K_{off} (1/s) | KD (M) | Kₒₙ (1/Ms) | K_{off} (1/s) | KD (M) |
| scFv-3B11 | 60 | 7.98 × 10³ | 3.85 × 10⁻⁴ | 4.82 × 10⁻⁸ | 9.82 × 10³ | 1.85 × 10⁻⁴ | 1.88 × 10⁻⁸ |
| scFv-1C5 | 60 | 2.52 × 10⁴ | 4.3 × 10⁻⁵ | 1.71 × 10⁻⁹ | 2.76 × 10⁴ | 3.49 × 10⁻⁵ | 1.26 × 10⁻⁹ |
| scFv-3C2 | 48 | 5.67 × 10³ | 2.55 × 10⁻⁴ | 4.49 × 10⁻⁸ | 8.36 × 10³ | 1.68 × 10⁻⁴ | 2 × 10⁻⁸ |
| scFv-4D2 | 60 | 1.19 × 10⁴ | 1.57 × 10⁻⁴ | 1.32 × 10⁻⁸ | 5.14 × 10³ | 1.2 × 10⁻⁴ | 2.33 × 10⁻⁸ |
| scFv-4D6 | 48 | 1.05 × 10⁴ | 1.93 × 10⁻⁴ | 1.84 × 10⁻⁸ | 1.15 × 10⁴ | 2.34 × 10⁻⁴ | 2.04 × 10⁻⁸ |
| scFv-3E11 | 24 | 1.52 × 10⁴ | 3.46 × 10⁻⁴ | 2.28 × 10⁻⁸ | 1.57 × 10⁴ | 4.84 × 10⁻⁴ | 3.09 × 10⁻⁸ |
| scFv-4F2 | 72 | 4.52 × 10³ | 1.37 × 10⁻⁴ | 3.07 × 10⁻⁸ | 5.69 × 10³ | 1.33 × 10⁻⁴ | 2.33 × 10⁻⁸ |
| scFv-5G8 | 60 | 6.35 × 10³ | 1.15 × 10⁻⁴ | 1.81 × 10⁻⁸ | 6.36 × 10³ | 2.21 × 10⁻⁴ | 3.48 × 10⁻⁸ |

### EXAMPLE 5

### Conversion of scFv to VH-Domain Antibodies

The regions corresponding to VH for each of the isolated antibodies were amplified by PCR with primers LW 196 and MKR 196 (5′-ATAGTACTCGAGCCCGATCCGGCCCCCGAGGC-3′) (SEQ ID NO:162) introducing an XhoI restriction site on the 3′-end. The amplified PCR product was subjected to restriction digestion with NcoI and XhoI and the fragments were subcloned into a bacterial expression vector with an N-terminal cleavable PeIB leader sequence for periplasmic expression and a C-terminal 6× His-tag for IMAC purification. The insertion of VH-domains was verified by sequencing of plasmid DNA from individual E. coli colonies. Soluble VH-domains were expressed and purified as described above. The binding affinity of VH-domains to HSA and cross-reactivity to MSA was determined with an Octet® Red system (FIG. 2B, FIG. 3C, and FIG. 3D). The summary of analysis is shown in Table 2.

TABLE 2
VH domain antibody binding to human (HSA) and mouse serum albumin (MSA).

| | Yields | HSA | | | MSA | | |
|---|---|---|---|---|---|---|---|
| | μg/L | Kₒₙ (1/Ms) | K_{off} (1/s) | KD (M) | Kₒₙ (1/Ms) | K_{off} (1/s) | KD (M) |
| VH-3B11 | 108 | 6.98 × 10³ | 1.26 × 10⁻⁴ | 1.81 × 10⁻⁸ | 8.2 × 10³ | 7.88 × 10⁻⁵ | 9.61 × 10⁻⁹ |
| VH-1C5 | 84 | 5.26 × 10³ | 9.58 × 10⁻⁵ | 1.82 × 10⁻⁸ | 8.57 × 10³ | 5.42 × 10⁻⁵ | 6.32 × 10⁻⁹ |
| VH-3C2 | 96 | 6.5 × 10³ | 1.55 × 10⁻⁴ | 2.38 × 10⁻⁸ | 6.87 × 10³ | 2.18 × 10⁻⁴ | 3.18 × 10⁻⁸ |
| VH-4D2 | 200 | 5.27 × 10³ | 3.17 × 10⁻⁴ | 6.01 × 10⁻⁸ | 5.17 × 10³ | 2.79 × 10⁻⁴ | 5.4 × 10⁻⁸ |
| VH-4D6 | 96 | 1.06 × 10⁴ | 1.41 × 10⁻⁴ | 1.33 × 10⁻⁸ | 8.34 × 10³ | 1.05 × 10⁻⁴ | 1.26 × 10⁻⁸ |
| VH-3E11 | 96 | 5.63 × 10³ | 1.33 × 10⁻⁴ | 2.36 × 10⁻⁸ | 5.75 × 10³ | 2.26 × 10⁻⁴ | 3.93 × 10⁻⁸ |
| VH-4F2 | 240 | 3.76 × 10³ | 1.3 × 10⁻⁴ | 3.46 × 10⁻⁸ | 4.71 × 10³ | 1.35 × 10⁻⁴ | 2.86 × 10⁻⁸ |
| VH-5G8 | 360 | 4.52 × 10³ | 1.08 × 10⁻⁴ | 2.4 × 10⁻⁸ | 5.01 × 10³ | 1.83 × 10⁻⁴ | 3.65 × 10⁻⁸ |

The invention is not limited to the embodiments described and exemplified above, but is capable of variation and modification within the scope of the appended claims.

We claim:

1. A heavy chain domain antibody that specifically binds to an epitope on serum albumin, comprising a heavy chain complementarity determining region (CDR) 1 having the amino acid sequence of SEQ ID NO:146 or SEQ ID NO:147, a heavy chain CDR2 having the amino acid sequence of SEQ ID NO:149 or SEQ ID NO:150, and a heavy chain CDR3 having an amino acid sequence having at least 95% sequence identity with an amino acid sequence selected from the group consisting of SEQ ID NO:8, SEQ ID NO:26, SEQ ID NO:44, SEQ ID NO:62, SEQ ID NO:80, SEQ ID NO:98, SEQ ID NO:116, and SEQ ID NO:134, wherein a heavy chain domain antibody comprising a heavy chain CDR3 having an amino acid sequence having less than 100% sequence identity with the amino acid sequence selected from the group consisting of SEQ ID NO:8, SEQ ID NO:26, SEQ ID NO:44, SEQ ID NO:62, SEQ ID NO:80, SEQ ID NO:98, SEQ ID NO:116, and SEQ ID NO:134 has serum albumin binding activity.

2. The heavy chain domain antibody of claim 1, wherein the antibody comprises a heavy chain framework (FWR) 1 having the amino acid sequence of SEQ ID NO: 145, a heavy chain FWR2 having the amino acid sequence of SEQ ID NO: 148 or SEQ ID NO:174, a heavy chain FWR3 having the amino acid sequence of SEQ ID NO: 151, and a heavy chain FWR4 having the amino acid sequence of SEQ ID NO: 152.

3. The heavy chain domain antibody of claim 1, wherein the serum albumin is human serum albumin or mouse serum albumin.

4. The heavy chain domain antibody of claim 1, wherein the serum albumin is human serum albumin.

5. The heavy chain domain antibody of claim 1, wherein the heavy chain comprises the amino acid sequence of SEQ ID NO:17.

6. The heavy chain domain antibody of claim 1, wherein the heavy chain comprises the amino acid sequence of SEQ ID NO:35.

7. The heavy chain domain antibody of claim 1, wherein the heavy chain comprises the amino acid sequence of SEQ ID NO:53.

8. The heavy chain domain antibody of claim 1, wherein the heavy chain comprises the amino acid sequence of SEQ ID NO:71.

9. The heavy chain domain antibody of claim 1, wherein the heavy chain comprises the amino acid sequence of SEQ ID NO:89.

10. The heavy chain domain antibody of claim 1, wherein the heavy chain comprises the amino acid sequence of SEQ ID NO:107.

11. The heavy chain domain antibody of claim 1, wherein the heavy chain comprises the amino acid sequence of SEQ ID NO:125.

12. The heavy chain domain antibody of claim 1, wherein the heavy chain comprises the amino acid sequence of SEQ ID NO:143.

13. The heavy chain domain antibody of claim 1, wherein the heavy chain domain antibody is conjugated to a detectable label, a chemotherapeutic agent, or biotin.

14. The heavy chain domain antibody of claim 1, wherein the affinity of the antibody for serum albumin is less than about 1×10⁻⁶ M.

15. The heavy chain domain antibody of claim 1, wherein the affinity of the antibody for serum albumin is less than about 1×10⁻⁸ M.

16. The heavy chain domain antibody of claim 1, wherein the affinity of the antibody for serum albumin is less than about 1×10⁻¹⁰ M.

17. A composition, comprising the heavy chain domain antibody of claim 1 and a pharmaceutically acceptable carrier.

18. An isolated polynucleotide encoding the heavy chain domain antibody of claim 1.

19. A vector, wherein the vector comprises the polynucleotide of claims 18.

20. A transformed cell, wherein the transformed cell comprises the vector of claim 19.

21. The transformed cell of claim 20, wherein the cell is a mammalian cell, a yeast cell, or a bacteria cell.

22. A method for enhancing the circulating half-life of the heavy chain domain antibody of claim 1, comprising contacting the heavy chain domain antibody with serum albumin in the blood of a subject.


SEQUENCE LISTING (188 sequences)

SEQ ID NO: 1 - DNA, 789 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacttgattgtagtggtggtagctgctactactactactacggtatggacgtctggggccaagggaccacggtcaccgtctcctcatcggcctcgggggccggatcgggcggcggcggctccggaggaggaggatctggtggtggtggttcgactagtgatattgtgatgacccacactccactctccctgcccgtcacccctggagagccggcctccatctcctgcaggtctagtcagagcctcttggatagtgatgatggaaacacctatttggactggtacctgcagaagccagggcagtctccacagctcctgatctatacgctttcctatcgggcctctggagtcccagacaggttcagtggcagtgggtcaggcactgattttacactgaaaatcagcagagtggaggctgacgatgttggcgtttattactgcatgcaagctctacaaactcctccagctttcggcggagggaccaaagtggagatcaaa

SEQ ID NO: 2 - protein, 263 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLDCSGGSCYYYYYGMDVWGQGTTVTVSSSASGAGSGGGGSGGGGSGGGGSTSDIVMTHTPLSLPVTPGEPASISCRSSQSLLDSDDGNTYLDWYLQKPGQSPQLLIYTLSYRASGVPDRFSGSGSGTDFTLKISRVEADDVGVYYCMQALQTPPAFGGGTKVEIK

SEQ ID NO: 3 - protein, 22 aa, Homo sapiens
EVQLVQSGAEVKKPGESLKISC

SEQ ID NO: 4 - protein, 13 aa, Homo sapiens
KGSGYSFTSYWIG

SEQ ID NO: 5 - protein, 14 aa, Homo sapiens
WVRQMPGKGLEWMG

SEQ ID NO: 6 - protein, 10 aa, Homo sapiens
IIYPGDSDTR

SEQ ID NO: 7 - protein, 37 aa, Homo sapiens
YSPSFQGQVTISADKSISTAYLQWSSLKASDTAMYYC

SEQ ID NO: 8 - protein, 19 aa, Homo sapiens
ARLDCSGGSCYYYYYGMDV

SEQ ID NO: 9 - protein, 18 aa, Homo sapiens
WGQGTTVTVSSSASGAGS

SEQ ID NO: 10 - protein, 25 aa, Homo sapiens
TSDIVMTHTPLSLPVTPGEPASISC

SEQ ID NO: 11 - protein, 17 aa, Homo sapiens
RSSQSLLDSDDGNTYLD

SEQ ID NO: 12 - protein, 14 aa, Homo sapiens
WYLQKPGQSPQLLI

SEQ ID NO: 13 - protein, 8 aa, Homo sapiens
YTLSYRAS

SEQ ID NO: 14 - protein, 32 aa, Homo sapiens
GVPDRFSGSGSGTDFTLKISRVEADDVGVYYC

SEQ ID NO: 15 - protein, 9 aa, Homo sapiens
MQALQTPPA

SEQ ID NO: 16 - protein, 10 aa, Homo sapiens
FGGGTKVEIK

SEQ ID NO: 17 - protein, 133 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLDCSGGSCYYYYYGMDVWGQGTTVTVSSSASGAGS

SEQ ID NO: 18 - protein, 115 aa, Homo sapiens
TSDIVMTHTPLSLPVTPGEPASISCRSSQSLLDSDDGNTYLDWYLQKPGQSPQLLIYTLSYRASGVPDRFSGSGSGTDFTLKISRVEADDVGVYYCMQALQTPPAFGGGTKVEIK

SEQ ID NO: 19 - DNA, 750 bp, Homo sapiens
cagatgcagctggtgcagtctggggctgaggtgaagaagcctgggacctcagtgaaggtctcctgcaaggcttctggatacaccttcaccagttatgatatcaactgggtgcgacaggccactggacaagggcttgagtgggtgggatggatgaaccctaacagaggtaacacaggttatgcacagaagttccaggaaagagtcaccattaccatggacatgtccacaagcacagcctacatggagctgagcagcctgagatctgaggacacggccgtgtattactgtgcgagagccatcccgggaggaggtgcttttgatatctggggccaagggaccacggtcaccgtctcctcatcggcctcgggggccggatcgggcggcggcggctccggaggaggaggatctggtggtggtggttcgactagtgaaacgacactcacgcagtctccagcattcatgtcagcgactccaggagacaaagtcaacatctcctgcaaagccagccaagacattgatgatgatatgaactggtaccaacagaaaccaggagaagctgctattttcattattcaagaagctactactctcgttcctggaatcccacctcgattcagtggcagcgggtatggaacagattttaccctcacaattaataacatagaatctgaggatgctgcatattacttctgtctacaacatgataatttcccgtggacgttcggccaagggaccaaagtggatatcaaa

SEQ ID NO: 20 - protein, 250 aa, Homo sapiens
QMQLVQSGAEVKKPGTSVKVSCKASGYTFTSYDINWVRQATGQGLEWVGWMNPNRGNTGYAQKFQERVTITMDMSTSTAYMELSSLRSEDTAVYYCARAIPGGGAFDIWGQGTTVTVSSSASGAGSGGGGSGGGGSGGGGSTSETTLTQSPAFMSATPGDKVNISCKASQDIDDDMNWYQQKPGEAAIFIIQEATTLVPGIPPRFSGSGYGTDFTLTINNIESEDAAYYFCLQHDNFPWTFGQGTKVDIK

SEQ ID NO: 21 - protein, 22 aa, Homo sapiens
QMQLVQSGAEVKKPGTSVKVSC

SEQ ID NO: 22 - protein, 13 aa, Homo sapiens
KASGYTFTSYDIN

SEQ ID NO: 23 - protein, 14 aa, Homo sapiens
WVRQATGQGLEWVG

SEQ ID NO: 24 - protein, 10 aa, Homo sapiens
WMNPNRGNTG

SEQ ID NO: 25 - protein, 37 aa, Homo sapiens
YAQKFQERVTITMDMSTSTAYMELSSLRSEDTAVYYC

SEQ ID NO: 26 - protein, 12 aa, Homo sapiens
ARAIPGGGAFDI

SEQ ID NO: 27 - protein, 18 aa, Homo sapiens
WGQGTTVTVSSSASGAGS

SEQ ID NO: 28 - protein, 25 aa, Homo sapiens
TSETTLTQSPAFMSATPGDKVNISC

SEQ ID NO: 29 - protein, 11 aa, Homo sapiens
KASQDIDDDMN

SEQ ID NO: 30 - protein, 14 aa, Homo sapiens
WYQQKPGEAAIFII

SEQ ID NO: 31 - protein, 8 aa, Homo sapiens
QEATTLVP

SEQ ID NO: 32 - protein, 32 aa, Homo sapiens
GIPPRFSGSGYGTDFTLTINNIESEDAAYYFC

SEQ ID NO: 33 - protein, 9 aa, Homo sapiens
LQHDNFPWT

SEQ ID NO: 34 - protein, 10 aa, Homo sapiens
FGQGTKVDIK

SEQ ID NO: 35 - protein, 126 aa, Homo sapiens
QMQLVQSGAEVKKPGTSVKVSCKASGYTFTSYDINWVRQATGQGLEWVGWMNPNRGNTGYAQKFQERVTITMDMSTSTAYMELSSLRSEDTAVYYCARAIPGGGAFDIWGQGTTVTVSSSASGAGS

SEQ ID NO: 36 - protein, 109 aa, Homo sapiens
TSETTLTQSPAFMSATPGDKVNISCKASQDIDDDMNWYQQKPGEAAIFIIQEATTLVPGIPPRFSGSGYGTDFTLTINNIESEDAAYYFCLQHDNFPWTFGQGTKVDIK

SEQ ID NO: 37 - DNA, 777 bp, Homo sapiens
caggtgcagctggtgcagtctggagctgaagtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagttccagggcagagtcaccatgaccgaggacacatctacagacacagcctacatggagctgagcagcctgagatctgaggacacggccgtgtattactgtgcaacagaggggtatagtggctacgatttatatgcttttgatatctggggccaagggacaatggtcaccgtctcttcatcggcctcgggggccggatcgggcggcggcggctccggaggaggaggatctggtggtggtggttcgactagtgatgttgtgatgactcagtctccactctccctgcccgtcacccctggagagccggcctccatctcctgcaggtctagtcagagcctcctgcatagtaatggatacaactatttgaattggtttcagcagaggccaggccagtctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggcctccgatcaccttcggccaagggacacgactggagattaaa

SEQ ID NO: 38 - protein, 259 aa, Homo sapiens
QVQLVQSGAEVKKPGASVKVSCKASGYTFTSYGISWVRQAPGQGLEWMGWISAYNGNTNYAQKFQGRVTMTEDTSTDTAYMELSSLRSEDTAVYYCATEGYSGYDLYAFDIWGQGTMVTVSSSASGAGSGGGGSGGGGSGGGGSTSDVVMTQSPLSLPVTPGEPASISCRSSQSLLHSNGYNYLNWFQQRPGQSPRRLIYKVSNRDSGVPDRFSGSGSGTDFTLKISRVEAEDVGVYYCMQGTHWPPITFGQGTRLEIK

SEQ ID NO: 39 - protein, 22 aa, Homo sapiens
QVQLVQSGAEVKKPGASVKVSC

SEQ ID NO: 40 - protein, 13 aa, Homo sapiens
KASGYTFTSYGIS

SEQ ID NO: 41 - protein, 14 aa, Homo sapiens
WVRQAPGQGLEWMG

SEQ ID NO: 42 - protein, 10 aa, Homo sapiens
WISAYNGNTN

SEQ ID NO: 43 - protein, 37 aa, Homo sapiens
YAQKFQGRVTMTEDTSTDTAYMELSSLRSEDTAVYYC

SEQ ID NO: 44 - protein, 15 aa, Homo sapiens
ATEGYSGYDLYAFDI

SEQ ID NO: 45 - protein, 18 aa, Homo sapiens
WGQGTMVTVSSSASGAGS

SEQ ID NO: 46 - protein, 25 aa, Homo sapiens
TSDVVMTQSPLSLPVTPGEPASISC

SEQ ID NO: 47 - protein, 16 aa, Homo sapiens
RSSQSLLHSNGYNYLN

SEQ ID NO: 48 - protein, 14 aa, Homo sapiens
WFQQRPGQSPRRLI

SEQ ID NO: 49 - protein, 8 aa, Homo sapiens
YKVSNRDS

SEQ ID NO: 50 - protein, 32 aa, Homo sapiens
GVPDRFSGSGSGTDFTLKISRVEAEDVGVYYC

SEQ ID NO: 51 - protein, 10 aa, Homo sapiens
MQGTHWPPIT

SEQ ID NO: 52 - protein, 10 aa, Homo sapiens
FGQGTRLEIK

SEQ ID NO: 53 - protein, 129 aa, Homo sapiens
QVQLVQSGAEVKKPGASVKVSCKASGYTFTSYGISWVRQAPGQGLEWMGWISAYNGNTNYAQKFQGRVTMTEDTSTDTAYMELSSLRSEDTAVYYCATEGYSGYDLYAFDIWGQGTMVTVSSSASGAGS

SEQ ID NO: 54 - protein, 115 aa, Homo sapiens
TSDVVMTQSPLSLPVTPGEPASISCRSSQSLLHSNGYNYLNWFQQRPGQSPRRLIYKVSNRDSGVPDRFSGSGSGTDFTLKISRVEAEDVGVYYCMQGTHWPPITFGQGTRLEIK

SEQ ID NO: 55 - DNA, 762 bp, Homo sapiens
gaggtccagctggtacagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcgcagaagttccagggcagagtcaccatgaccagggacacgtccacgagcacagtctacatggagctgagcagcctgaggcctgaggacacggccgtgtattactgtgcgagagagcgtcgttactatggttcggggagttattataagtttgtctactggggccagggaaccctggtcaccgtctcctcatcggcctcgggggccggatcgggcggcggcggctccggaggaggaggatctggtggtggtggttcgactagtaattttatgctgactcagccccactctgtgtcggagtctccggggaagacggtaaccatctcctgcaccggcagcagtggtcgcattgacagcaactatgtacagtggtaccagcagcgcccgggcagtgcccccaccactgtgatctatgaagatacccaaagaccctctggggtccctgatcgcttctctggctccctcgacagttcctccaactctgcctccctcaccatctctgggctgaagactgaggacgaggctgactactactgtcagtctctttgggtgttcggcggagggaccaaggtcaccgtgcta

SEQ ID NO: 56 - protein, 254 aa, Homo sapiens
EVQLVQSGAEVKKPGASVKVSCKASGYTFTSYYMHWVRQAPGQGLEWMGIINPSGGSTSYAQKFQGRVTMTRDTSTSTVYMELSSLRPEDTAVYYCARERRYYGSGSYYKFVYWGQGTLVTVSSSASGAGSGGGGSGGGGSGGGGSTSNFMLTQPHSVSESPGKTVTISCTGSSGRIDSNYVQWYQQRPGSAPTTVIYEDTQRPSGVPDRFSGSLDSSSNSASLTISGLKTEDEADYYCQSLWVFGGGTKVTVL

SEQ ID NO: 57 - protein, 22 aa, Homo sapiens
EVQLVQSGAEVKKPGASVKVSC

SEQ ID NO: 58 - protein, 13 aa, Homo sapiens
KASGYTFTSYYMH

SEQ ID NO: 59 - protein, 14 aa, Homo sapiens
WVRQAPGQGLEWMG

SEQ ID NO: 60 - protein, 10 aa, Homo sapiens
IINPSGGSTS

SEQ ID NO: 61 - protein, 37 aa, Homo sapiens
YAQKFQGRVTMTRDTSTSTVYMELSSLRPEDTAVYYC

SEQ ID NO: 62 - protein, 17 aa, Homo sapiens
ARERRYYGSGSYYKFVY

SEQ ID NO: 63 - protein, 18 aa, Homo sapiens
WGQGTLVTVSSSASGAGS

SEQ ID NO: 64 - protein, 24 aa, Homo sapiens
TSNFMLTQPHSVSESPGKTVTISC

SEQ ID NO: 65 - protein, 13 aa, Homo sapiens
TGSSGRIDSNYVQ

SEQ ID NO: 66 - protein, 14 aa, Homo sapiens
WYQQRPGSAPTTVI

SEQ ID NO: 67 - protein, 8 aa, Homo sapiens
YEDTQRPS

SEQ ID NO: 68 - protein, 34 aa, Homo sapiens
GVPDRFSGSLDSSSNSASLTISGLKTEDEADYYC

SEQ ID NO: 69 - protein, 5 aa, Homo sapiens
QSLWV

SEQ ID NO: 70 - protein, 10 aa, Homo sapiens
FGGGTKVTVL

SEQ ID NO: 71 - protein, 131 aa, Homo sapiens
EVQLVQSGAEVKKPGASVKVSCKASGYTFTSYYMHWVRQAPGQGLEWMGIINPSGGSTSYAQKFQGRVTMTRDTSTSTVYMELSSLRPEDTAVYYCARERRYYGSGSYYKFVYWGQGTLVTVSSSASGAGS

SEQ ID NO: 72 - protein, 108 aa, Homo sapiens
TSNFMLTQPHSVSESPGKTVTISCTGSSGRIDSNYVQWYQQRPGSAPTTVIYEDTQRPSGVPDRFSGSLDSSSNSASLTISGLKTEDEADYYCQSLWVFGGGTKVTVL

SEQ ID NO: 73 - DNA, 768 bp, Homo sapiens
caggtacagctgcagcagtcaggtccaggactggtgaagccctcgcagaccctctcactcacctgtgccatctccggggacagtgtctctggcaacagtgctgcttggaactggatcaggcagtccccatcgagaggccttgagtggctgggaaggacatactacaggtccaagtggtatattggttatgcagcatctctgcaaagtcgattagccatcaacgcagacacgtccaagaaccagttgtccctgcgtctgagctctgtcactcccgaggacacggctgtttattactgcgtaggccgacactacagtgggcactcgggtgcttttgatacctggggccatgggacaatggtcaccgtctcttcatcggcctcgggggccggatcgggcggcggcggctccggaggaggaggatctggtggtggtggttcgactagtgaaattgtgttgacgcagtctccaggcaccctgtctttgtctccaggggaaagagccaccctctcctgcagggccagtcagagtgttatcagcaaccacttagcctggtaccagcagaaacctggccaggctcctaggctcctcatctatggtgcatccagcagggccactggcatcccagacaggttcagtggcagtgggtctgggacagacttcactcttaccatcaacagactggagcctgaagattttgcagtgtattactgtcagcagtatggtacctcaccgatcaccttcggccaagggacacgactggagattaaa

SEQ ID NO: 74 - protein, 256 aa, Homo sapiens
QVQLQQSGPGLVKPSQTLSLTCAISGDSVSGNSAAWNWIRQSPSRGLEWLGRTYYRSKWYIGYAASLQSRLAINADTSKNQLSLRLSSVTPEDTAVYYCVGRHYSGHSGAFDTWGHGTMVTVSSSASGAGSGGGGSGGGGSGGGGSTSEIVLTQSPGTLSLSPGERATLSCRASQSVISNHLAWYQQKPGQAPRLLIYGASSRATGIPDRFSGSGSGTDFTLTINRLEPEDFAVYYCQQYGTSPITFGQGTRLEIK

SEQ ID NO: 75 - protein, 22 aa, Homo sapiens
QVQLQQSGPGLVKPSQTLSLTC

SEQ ID NO: 76 - protein, 15 aa, Homo sapiens
AISGDSVSGNSAAWN

SEQ ID NO: 77 - protein, 14 aa, Homo sapiens
WIRQSPSRGLEWLG

SEQ ID NO: 78 - protein, 11 aa, Homo sapiens
RTYYRSKWYIG

SEQ ID NO: 79 - protein, 37 aa, Homo sapiens
YAASLQSRLAINADTSKNQLSLRLSSVTPEDTAVYYC

SEQ ID NO: 80 - protein, 14 aa, Homo sapiens
VGRHYSGHSGAFDT

SEQ ID NO: 81 - protein, 18 aa, Homo sapiens
WGHGTMVTVSSSASGAGS

SEQ ID NO: 82 - protein, 25 aa, Homo sapiens
TSEIVLTQSPGTLSLSPGERATLSC

SEQ ID NO: 83 - protein, 12 aa, Homo sapiens
RASQSVISNHLA

SEQ ID NO: 84 - protein, 14 aa, Homo sapiens
WYQQKPGQAPRLLI

SEQ ID NO: 85 - protein, 8 aa, Homo sapiens
YGASSRAT

SEQ ID NO: 86 - protein, 32 aa, Homo sapiens
GIPDRFSGSGSGTDFTLTINRLEPEDFAVYYC

SEQ ID NO: 87 - protein, 9 aa, Homo sapiens
QQYGTSPIT

SEQ ID NO: 88 - protein, 10 aa, Homo sapiens
FGQGTRLEIK

SEQ ID NO: 89 - protein, 131 aa, Homo sapiens
QVQLQQSGPGLVKPSQTLSLTCAISGDSVSGNSAAWNWIRQSPSRGLEWLGRTYYRSKWYIGYAASLQSRLAINADTSKNQLSLRLSSVTPEDTAVYYCVGRHYSGHSGAFDTWGHGTMVTVSSSASGAGS

SEQ ID NO: 90 - protein, 110 aa, Homo sapiens
TSEIVLTQSPGTLSLSPGERATLSCRASQSVISNHLAWYQQKPGQAPRLLIYGASSRATGIPDRFSGSGSGTDFTLTINRLEPEDFAVYYCQQYGTSPITFGQGTRLEIK

SEQ ID NO: 91 - DNA, 784 bp, Homo sapiens
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcgtctatcctggtgactctgataccagatacagcccgtccttccaaggccaggtcaccatctcagccggcaagtccatcagcaccgcctacctgcagtggagcagcctgaaggcctcggacaccgccatgtattactgtgcgagaccaagtatgggagctactacgtggattagtgcttttgatatctggggccaagggaccacggtcaccgtctcctcatcggcctcgggggccggatcgggcggcggcggctccggaggaggaggatctggtggtggtggttcgactagtgatgttgtgatgactcagtctccactctccctgcccgtcacccctggagagccggcctccatctcctgcaggtctagtcagagcctcctgcatagtaatggatacaactatttggattggtacctgcagaagccaggccagtctccacagctcgtgatctatgaagtttccactcggctccctggagtgtcagataggttcagtggcagcgggtcagggacagatttcacactgaaaatcagccgggtggaggctgaggatgttggggtttattactgcatgcaaactagacagcttccgtacgcttttgggccaggggaccaaggtggatatcaaacgctcg

SEQ ID NO: 92 - protein, 261 aa, Homo sapiens
EVQLVQSGAEVKKPGESLKISCKGSGYSFTSYWIGWVRQMPGKGLEWMGIVYPGDSDTRYSPSFQGQVTISAGKSISTAYLQWSSLKASDTAMYYCARPSMGATTWISAFDIWGQGTTVTVSSSASGAGSGGGGSGGGGSGGGGSTSDVVMTQSPLSLPVTPGEPASISCRSSQSLLHSNGYNYLDWYLQKPGQSPQLVIYEVSTRLPGVSDRFSGSGSGTDFTLKISRVEAEDVGVYYCMQTRQLPYAFGPGDQGGYQTL

SEQ ID NO: 93 - protein, 22 aa, Homo sapiens
EVQLVQSGAEVKKPGESLKISC

SEQ ID NO: 94 - protein, 13 aa, Homo sapiens
KGSGYSFTSYWIG

SEQ ID NO: 95 - protein, 14 aa, Homo sapiens
WVRQMPGKGLEWMG

SEQ ID NO: 96 - protein, 10 aa, Homo sapiens
IVYPGDSDTR

SEQ ID NO: 97 - protein, 37 aa, Homo sapiens
YSPSFQGQVTISAGKSISTAYLQWSSLKASDTAMYYC

SEQ ID NO: 98 - protein, 16 aa, Homo sapiens
ARPSMGATTWISAFDI

SEQ ID NO: 99 - protein, 18 aa, Homo sapiens
WGQGTTVTVSSSASGAGS

SEQ ID NO: 100 - protein, 25 aa, Homo sapiens
TSDVVMTQSPLSLPVTPGEPASISC

SEQ ID NO: 101 - protein, 16 aa, Homo sapiens
RSSQSLLHSNGYNYLD

SEQ ID NO: 102 - protein, 14 aa, Homo sapiens
WYLQKPGQSPQLVI

SEQ ID NO: 103 - protein, 8 aa, Homo sapiens
YEVSTRLP

SEQ ID NO: 104 - protein, 32 aa, Homo sapiens
GVSDRFSGSGSGTDFTLKISRVEAEDVGVYYC

SEQ ID NO: 105 - protein, 9 aa, Homo sapiens
MQTRQLPYA

SEQ ID NO: 106 - protein, 12 aa, Homo sapiens
FGPGDQGGYQTL

SEQ ID NO: 107 - protein, 130 aa, Homo sapiens
EVQLVQSGAEVKKPGESLKISCKGSGYSFTSYWIGWVRQMPGKGLEWMGIVYPGDSDTRYSPSFQGQVTISAGKSISTAYLQWSSLKASDTAMYYCARPSMGATTWISAFDIWGQGTTVTVSSSASGAGS

SEQ ID NO: 108 - protein, 116 aa, Homo sapiens
TSDVVMTQSPLSLPVTPGEPASISCRSSQSLLHSNGYNYLDWYLQKPGQSPQLVIYEVSTRLPGVSDRFSGSGSGTDFTLKISRVEAEDVGVYYCMQTRQLPYAFGPGDQGGYQTL

SEQ ID NO: 109 - DNA, 750 bp, Homo sapiens
caggtacagctgcagcagtcaggtccaggactggtgaagccctcgcagaccctctcactcacctgtgccatctccggggacagtgtttctagtaaaagtgctgcttggaactggatcaggcagtccccatcgagaggccttgagtggctgggaaggacatactacaggtccaagtggtataatgattatgtagtatctctgagaagtcgaataaccgtcagcccagacacatctaagaaccagttctccctgcagctgaactctgtgactcccgaggacacggctatgtattattgtgcaaggacaagagggcacatcgatctctggggccgtggaaccctggtcaccgtctcctcatcggcctcgggggccggatcgggcggcggcggctccggaggaggaggatctggtggtggtggttcgactagtgacatccagttgacccagtctccatcttctgtgtctgcatctgtaggagacagagtcatcatcacttgtcgggcgagtcagggtattggcaggtggttagcctggtatcagcagaaaccagggaaagcccctaagctcctgatctatgctgcatccagtttgcaaagtggggtcccatcaaggttcagtggcagtggatctgggtcagatttcactctcaccatcagcagtctgcaacctgaagattttgcaacttactactgtcaacagagttacagtaccccgctcactttcggcggagggaccaaactggaaatcaaa

SEQ ID NO: 110 - protein, 250 aa, Homo sapiens
QVQLQQSGPGLVKPSQTLSLTCAISGDSVSSKSAAWNWIRQSPSRGLEWLGRTYYRSKWYNDYVVSLRSRITVSPDTSKNQFSLQLNSVTPEDTAMYYCARTRGHIDLWGRGTLVTVSSSASGAGSGGGGSGGGGSGGGGSTSDIQLTQSPSSVSASVGDRVIITCRASQGIGRWLAWYQQKPGKAPKLLIYAASSLQSGVPSRFSGSGSGSDFTLTISSLQPEDFATYYCQQSYSTPLTFGGGTKLEIK

SEQ ID NO: 111 - protein, 22 aa, Homo sapiens
QVQLQQSGPGLVKPSQTLSLTC

SEQ ID NO: 112 - protein, 15 aa, Homo sapiens
AISGDSVSSKSAAWN

SEQ ID NO: 113 - protein, 14 aa, Homo sapiens
WIRQSPSRGLEWLG

SEQ ID NO: 114 - protein, 11 aa, Homo sapiens
RTYYRSKWYND

SEQ ID NO: 115 - protein, 37 aa, Homo sapiens
YVVSLRSRITVSPDTSKNQFSLQLNSVTPEDTAMYYC

SEQ ID NO: 116 - protein, 9 aa, Homo sapiens
ARTRGHIDL

SEQ ID NO: 117 - protein, 18 aa, Homo sapiens
WGRGTLVTVSSSASGAGS

SEQ ID NO: 118 - protein, 25 aa, Homo sapiens
TSDIQLTQSPSSVSASVGDRVIITC

SEQ ID NO: 119 - protein, 11 aa, Homo sapiens
RASQGIGRWLA

SEQ ID NO: 120 - protein, 14 aa, Homo sapiens
WYQQKPGKAPKLLI

SEQ ID NO: 121 - protein, 8 aa, Homo sapiens
YAASSLQS

SEQ ID NO: 122 - protein, 32 aa, Homo sapiens
GVPSRFSGSGSGSDFTLTISSLQPEDFATYYC

SEQ ID NO: 123 - protein, 9 aa, Homo sapiens
QQSYSTPLT

SEQ ID NO: 124 - protein, 10 aa, Homo sapiens
FGGGTKLEIK

SEQ ID NO: 125 - protein, 126 aa, Homo sapiens
QVQLQQSGPGLVKPSQTLSLTCAISGDSVSSKSAAWNWIRQSPSRGLEWLGRTYYRSKWYNDYVVSLRSRITVSPDTSKNQFSLQLNSVTPEDTAMYYCARTRGHIDLWGRGTLVTVSSSASGAGS

SEQ ID NO: 126 - protein, 109 aa, Homo sapiens
TSDIQLTQSPSSVSASVGDRVIITCRASQGIGRWLAWYQQKPGKAPKLLIYAASSLQSGVPSRFSGSGSGSDFTLTISSLQPEDFATYYCQQSYSTPLTFGGGTKLEIK

SEQ ID NO: 127 - DNA, 771 bp, Homo sapiens
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagaactccctcaaccggtacacttgactgctggggccggggcaccctggtcaccgtctcctcatcggcctcgggggccggatcgggcggcggcggctccggaggaggaggatctggtggtggtggttcgactagtgatgttgtgatgactcagtctccactctccctgcccgtcacccttggacagccggcctccatctcctgcaggtctagtcaaagcctcgtacacagtgatggaaacacctacttgaattggtttcagcagaggccaggccaatctccaaggcgcctaatttataaggtttctaaccgggactctggggtcccggacagattcagcggcagtgggtcaggcactgatttcacactggaaatcagcagggtggaggctgaggatgttggggtttattactgcatgcaagctacacactccccatacagtttcggccctgggaccaaactggaaatcaaa

SEQ ID NO: 128 - protein, 257 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARTPSTGTLDCWGRGTLVTVSSSASGAGSGGGGSGGGGSGGGGSTSDVVMTQSPLSLPVTLGQPASISCRSSQSLVHSDGNTYLNWFQQRPGQSPRRLIYKVSNRDSGVPDRFSGSGSGTDFTLEISRVEAEDVGVYYCMQATHSPYSFGPGTKLEIK

SEQ ID NO: 129 - protein, 22 aa, Homo sapiens
QVQLQQSGPGLVKPSQTLSLTC

SEQ ID NO: 130 - protein, 15 aa, Homo sapiens
AISGDSVSSNSAAWN

SEQ ID NO: 131 - protein, 14 aa, Homo sapiens
WIRQSPSRGLEWLG

SEQ ID NO: 132 - protein, 11 aa, Homo sapiens
RTYYRSKWYND

SEQ ID NO: 133 - protein, 37 aa, Homo sapiens
YAVSVKSRITINPDTSKNQFSLQLNSVTPEDTAVYYC

SEQ ID NO: 134 - protein, 11 aa, Homo sapiens
ARTPSTGTLDC

SEQ ID NO: 135 - protein, 18 aa, Homo sapiens
WGRGTLVTVSSSASGAGS

SEQ ID NO: 136 - protein, 25 aa, Homo sapiens
TSDVVMTQSPLSLPVTLGQPASISC

SEQ ID NO: 137 - protein, 16 aa, Homo sapiens
RSSQSLVHSDGNTYLN

SEQ ID NO: 138 - protein, 14 aa, Homo sapiens
WFQQRPGQSPRRLI

SEQ ID NO: 139 - protein, 8 aa, Homo sapiens
YKVSNRDS

SEQ ID NO: 140 - protein, 32 aa, Homo sapiens
GVPDRFSGSGSGTDFTLEISRVEAEDVGVYYC

SEQ ID NO: 141 - protein, 9 aa, Homo sapiens
MQATHSPYS

SEQ ID NO: 142 - protein, 10 aa, Homo sapiens
FGPGTKLEIK

SEQ ID NO: 143 - protein, 128 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARTPSTGTLDCWGRGTLVTVSSSASGAGS

SEQ ID NO: 144 - protein, 114 aa, Homo sapiens
TSDVVMTQSPLSLPVTLGQPASISCRSSQSLVHSDGNTYLNWFQQRPGQSPRRLIYKVSNRDSGVPDRFSGSGSGTDFTLEISRVEAEDVGVYYCMQATHSPYSFGPGTKLEIK

SEQ ID NO: 145 - protein, 22 aa, Homo sapiens
  VARIANT (1)..(22): X1 is E or Q; X2 is V or M; X5 is Q or V; X9 is A or P; X10 is E or G; X11 is L or V; X12 is K or V; X15 is G or S; X16 is E, A, Q or T; X17 is S or T; X18 is L or V; X19 is K or S; X20 is I, V or L; X21 is S or T
XXQLXQSGXXXXKPXXXXXXXC

SEQ ID NO: 146 - protein, 15 aa, Homo sapiens
  VARIANT (1)..(15): X9 is S or G; X10 is N or K
AISGDSVSXXSAAWN

SEQ ID NO: 147 - protein, 13 aa, Homo sapiens
  VARIANT (1)..(13): X2 is G or A; X6 is S or T; X11 is W, G, D or Y; X12 is I or M; X13 is G, S, N or H
KXSGYXFTSYXXX

SEQ ID NO: 148 - protein, 14 aa, Homo sapiens
  VARIANT (1)..(14): X2 is V or I; X5 is M, A or S; X6 is P or T; X7 is G or S; X8 is K, Q or R; X13 is M, L or V
WXRQXXXXGLEWXG

SEQ ID NO: 149 - protein, 11 aa, Homo sapiens
  VARIANT (1)..(11): X10 is N or I; X11 is D or G
RTYYRSKWYXX

SEQ ID NO: 150 - protein, 10 aa, Homo sapiens
  VARIANT (1)..(10): X1 is Y or W; X2 is I, V or M; X3 is Y, S or N; X4 is P or A; X5 is G, Y, N or S; X6 is D, N, R or G; X7 is S or G; X8 is D, N or S; X10 is R, N, G or S
XXXXXXXXTX

SEQ ID NO: 151 - protein, 37 aa, Homo sapiens
  VARIANT (1)..(37): X2 is S, A or V; X3 is P, Q, V or A; X4 is S or K; X5 is F, V or L; X6 is Q, K or R; X7 is G, S or E; X8 is Q or R; X9 is V, I or L; X10 is T or A; X11 is I, M or V; X12 is S, T or N; X13 is A, E, P, M or R; X14 is D or G; X15 is K, T or M; X17 is I, T or
YXXXXXXXXXXXXXXSXXXXXXXXXSXXXXDTAXYYC

SEQ ID NO: 152 - protein, 18 aa, Homo sapiens
  VARIANT (1)..(18): X3 is Q, R or H; X6 is T, M or L
WGXGTXVTVSSSASGAGS

SEQ ID NO: 153 - protein, 21 aa, Homo sapiens
  VARIANT (1)..(21): X3 is D, E or N; X4 is I, V, T or F; X5 is V, Q, T or M; X6 is M or L; X8 is H or Q; X9 is T, S or P; X10 is P or H; X11 is L, G, S or A; X12 is S, T, F or V; X13 is L, V, M or S; X14 is P, S or E; X15 is V, L, A or S; X16 is T, S or P; X17 is P, L, V or G;
TSXXXXTXXXXXXXXXXXXXX

SEQ ID NO: 154 - protein, 12 aa, Homo sapiens
  VARIANT (1)..(12): X1 is R, K or T; X2 is S, A or G; X4 is Q or S; X5 is S, G or D; X6 is L, V, I or R; X7 is L, V, I, G or D; X8 is D, H, S or R; X9 is S, N, W or D; X10 is D, N, H, L or M; X11 is D, G, L, A, N or Y; X12 is G, Y, N, A, or V
XXSXXXXXXXXX

SEQ ID NO: 155 - protein, 14 aa, Homo sapiens
  VARIANT (1)..(14): X2 is Y or F; X3 is L or Q; X5 is K or R; X8 is Q, K, E or S; X9 is S or A; X10 is P or A; X11 is Q, R, K, I or T; X12 is L, R, F or T; X13 is L, V or I
WXXQXPGXXXXXXI

SEQ ID NO: 156 - protein, 8 aa, Homo sapiens
  VARIANT (1)..(8): X1 is Y or Q; X2 is T, E, K, G, or A; X3 is L, V, A or D; X4 is S or T; X5 is Y, T, N, S or Q; X6 is R or L; X7 is A, L, D, Q, V or P; X8 is S, P or T
XXXXXXXX

SEQ ID NO: 157 - protein, 32 aa, Homo sapiens
  VARIANT (1)..(32): X2 is V or I; X3 is P or S; X4 is G or L; X10 is G or L; X11 is S, Y or D; X12 is G or S; X13 is T or S; X14 is D or S; X15 is F or N; X16 is T or S; X17 is L or A; X18 is K, E, T or S; X19 is I or L; X20 is S, N or T; X21 is R, S, N or I; X22 is V, L, I or S;
GXXXRFSGSXXXXXXXXXXXXXXXXXXXXXXX

SEQ ID NO: 158 - protein, 6 aa, Homo sapiens
  VARIANT (1)..(6): X1 is M, Q or L; X2 is Q or S; X3 is A, T, G, Y, S, H or L; X4 is L, R, T, G, Y, D or W; X5 is Q, H, T, S, N or V; X6 is T, L, W, S, or F
XXXXXX

SEQ ID NO: 159 - protein, 10 aa, Homo sapiens
  VARIANT (1)..(10): X3 is G, P or Q; X5 is T or D; X6 is K, Q or R; X7 is V, G or L; X8 is E, G, D or T; X9 is I, Y or V; X10 is K, Q or L
FGXGXXXXXX

SEQ ID NO: 160 - DNA, 17 bp, Homo sapiens
caggaaacagctatgac

SEQ ID NO: 161 - DNA, 18 bp, Homo sapiens
gttgggaagggcgatcgg

SEQ ID NO: 162 - DNA, 32 bp, Homo sapiens
atagtactcgagcccgatccggcccccgaggc

SEQ ID NO: 163 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 229 aa, Homo sapiens
SPNMVPHAHHAQAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 166 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 167 - protein, 5 aa, Homo sapiens
GGGGS

SEQ ID NO: 168 - protein, 10 aa, Homo sapiens
GGGGSGGGGS

SEQ ID NO: 169 - protein, 15 aa, Homo sapiens
GGGGSGGGGSGGGGS

SEQ ID NO: 170 - protein, 20 aa, Homo sapiens
GGGGSGGGGSGGGGSGGGGS

SEQ ID NO: 171 - protein, 25 aa, Homo sapiens
GGGGSGGGGSGGGGSGGGGSGGGGS

SEQ ID NO: 172 - protein, 19 aa, Homo sapiens
GGGGSKKGGGGSKKGGGGS

SEQ ID NO: 173 - protein, 17 aa, Homo sapiens
GGGGSKGGGGSKGGGGS

SEQ ID NO: 174 - protein, 14 aa, Homo sapiens
  VARIANT (1)..(14): X2 is V, I or F; X5 is M, A or S; X6 is P or T; X7 is G or S; X8 is K, Q or R; X9 is G or E; X10 is L or R; X12 is W, G, or I; X13 is M, L or V
WXRQXXXXXXEXXG

SEQ ID NO: 175 - protein, 22 aa, Homo sapiens
  VARIANT (1)..(22): X3 is D, E or N; X4 is I, V, T or F; X5 is V, Q, T or M; X6 is M or L; X8 is H or Q; X9 is T, S or P; X10 is P or H; X11 is L, G, S or A; X12 is S, T, F or V; X13 is L, V, M or S; X14 is P, S or E; X15 is V, L, A or S; X16 is T, S or P; X17 is P, L, V or
TSXXXXTXXXXXXXXXXXXXXC

SEQ ID NO: 176 - protein, 13 aa, Homo sapiens
  VARIANT (1)..(13): X1 is R, K or T; X2 is S, A or G; X4 is Q or S; X5 is S, G or D; X6 is L, V, I or R; X7 is L, V, I, G or D; X8 is D, H, S or R; X9 is S, N, W or D; X10 is D, N, H, L or M; X11 is D, G, L, A, N or Y; X12 is G, Y, N, A or V; X13 is N, T, or Q
XXSXXXXXXXXXX

SEQ ID NO: 177 - protein, 14 aa, Homo sapiens
  VARIANT (1)..(14): X1 is R, K or T; X2 is S, A or G; X4 is Q or S; X5 is S, G or D; X6 is L, V, I or R; X7 is L, V, I, G or D; X8 is D, H, S or R; X9 is S, N, W or D; X10 is D, N, H, L or M; X11 is D, G, L, A, N or Y; X12 is G, Y, N, A or V; X13 is N, T or Q; X14 is T or Y
XXSXXXXXXXXXXX

SEQ ID NO: 178 - protein, 15 aa, Homo sapiens
  VARIANT (1)..(15): X1 is R, K or T; X2 is S, A or G; X4 is Q or S; X5 is S, G or D; X6 is L, V, I or R; X7 is L, V, I, G or D; X8 is D, H, S or R; X9 is S, N, W or D; X10 is D, N, H, L or M; X11 is D, G, L, A, N or Y; X12 is G, Y, N, A or V; X13 is N, T or Q; X14 is T or Y; X15
XXSXXXXXXXXXXXX

SEQ ID NO: 179 - protein, 16 aa, Homo sapiens
  VARIANT (1)..(16): X1 is R, K or T; X2 is S, A or G; X4 is Q or S; X5 is S, G or D; X6 is L, V, I or R; X7 is L, V, I, G or D; X8 is D, H, S or R; X9 is S, N, W or D; X10 is D, N, H, L or M; X11 is D, G, L, A, N or Y; X12 is G, Y, N, A or V; X13 is N, T or Q; X14 is T or Y; X15
XXSXXXXXXXXXXXXX

SEQ ID NO: 180 - protein, 17 aa, Homo sapiens
  VARIANT (1)..(17): X1 is R, K or T; X2 is S, A or G; X4 is Q or S; X5 is S, G or D; X6 is L, V, I or R; X7 is L, V, I, G or D; X8 is D, H, S or R; X9 is S, N, W or D; X10 is D, N, H, L or M; X11 is D, G, L, A, N or Y; X12 is G, Y, N, A or V; X13 is N, T or Q; X14 is T or Y; X15
XXSXXXXXXXXXXXXXD

SEQ ID NO: 181 - protein, 34 aa, Homo sapiens
  VARIANT (1)..(34): X2 is V or I; X3 is P or S; X4 is G or L; X10 is G or L; X11 is S, Y or D; X12 is G or S; X13 is T or S; X14 is D or S; X15 is F or N; X16 is T or S; X17 is L or A; X18 is K, E, T or S; X19 is I or L; X20 is S, N or T; X21 is R, S, N or I; X22 is V, L, I or S;
GXXXRFSGSXXXXXXXXXXXXXXXXXXXXXXXYC

SEQ ID NO: 182 - protein, 33 aa, Homo sapiens
  VARIANT (1)..(33): X2 is V or I; X3 is P or S; X4 is G or L; X10 is G or L; X11 is S, Y or D; X12 is G or S; X13 is T or S; X14 is D or S; X15 is F or N; X16 is T or S; X17 is L or A; X18 is K, E, T or S; X19 is I or L; X20 is S, N or T; X21 is R, S, N or I; X22 is V, L, I or S;
GXXXRFSGSXXXXXXXXXXXXXXXXXXXXXXXY

SEQ ID NO: 183 - protein, 7 aa, Homo sapiens
  VARIANT (1)..(7): X1 is M, Q or L; X2 is Q or S; X3 is A, T, G, Y, S, H or L; X4 is L, R, T, G, Y, D or W; X5 is Q, H, T, S, N or V; X6 is T, L, W, S or F
XXXXXXP

SEQ ID NO: 184 - protein, 8 aa, Homo sapiens
  VARIANT (1)..(8): X1 is M, Q or L; X2 is Q or S; X3 is A, T, G, Y, S, H or L; X4 is L, R, T, G, Y, D or W; X5 is Q, H, T, S, N or V; X6 is T, L, W, S or F;  X8 is P, Y, I, L or W
XXXXXXPX

SEQ ID NO: 185 - protein, 9 aa, Homo sapiens
  VARIANT (1)..(9): X1 is M, Q or L; X2 is Q or S; X3 is A, T, G, Y, S, H or L; X4 is L, R, T, G, Y, D or W; X5 is Q, H, T, S, N or V; X6 is T, L, W, S, or F; X8 is P, Y, I, L or W; X9 is A, I, S or T
XXXXXXPXX

SEQ ID NO: 186 - protein, 10 aa, Homo sapiens
  VARIANT (1)..(10): X1 is M, Q or L; X2 is Q or S; X3 is A, T, G, Y, S, H or L; X4 is L, R, T, G, Y, D or W; X5 is Q, H, T, S, N or V; X6 is T, L, W, S, or F; X8 is P, Y, I, L or W; X9 is A, I, S or T
XXXXXXPXXT

SEQ ID NO: 187 - protein, 11 aa, Homo sapiens
  VARIANT (1)..(11): X3 is G, P or Q; X5 is T or D; X6 is K, Q or R; X7 is V, G or L; X8 is E, G, D or T; X9 is I, Y or V; X10 is K, Q or L
FGXGXXXXXXT

SEQ ID NO: 188 - protein, 12 aa, Homo sapiens
  VARIANT (1)..(12): X3 is G, P or Q; X5 is T or D; X6 is K, Q or R; X7 is V, G or L; X8 is E, G, D or T; X9 is I, Y or V; X10 is K, Q or L
FGXGXXXXXXTL